(19) United States
(12) Patent Application Publication
(10) Pub. No.: US 2006/0069095 A1
(43) Pub. Date: Mar. 30, 2006

(54) Azole derivatives

(76) Inventors: Thomas Hofmeister, Biblis (DE); Ulrike Reiff, Penzberg (DE); Thomas von Hirschheydt, Penzberg (DE); Edgar Voss, Bichl (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/232,303

(22) Filed: Sep. 21, 2005

(30) Foreign Application Priority Data
Sep. 24, 2004 (EP) 04022754.8

(51) Int. Cl. A61K 31/5377; A61K 31/422; C07D 413/02; C07D 413/14

(52) U.S. Cl. 514/235.2; 514/374; 544/137; 548/235

(57) ABSTRACT

Compounds of formula I 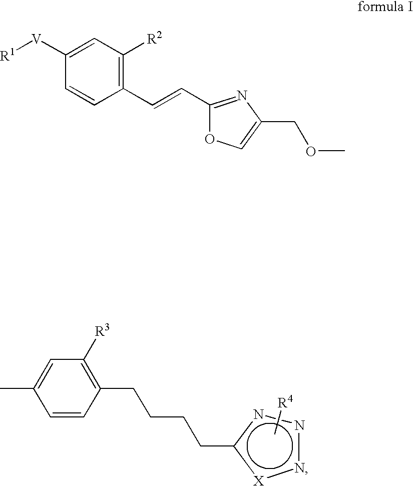 their pharmaceutically acceptable salts, enantiomeric forms, diastereoisomers and racemates, are disclosed. Also disclosed are methods of preparation of the above-mentioned compounds, pharmaceutical compositions and salts and esters thereof containing them, as well as the use of the above-mentioned compounds in the treatment, control or prevention of illnesses such as cancer.

### FIELD OF THE INVENTION

The present invention relates to novel azole derivatives, to a process for their manufacture, pharmaceutical compositions containing them, their manufacture, and the use of these compounds as pharmaceutically active agents useful in the treatment of cancer.

### BACKGROUND OF THE INVENTION

Protein tyrosine kinases (PTKs) catalyze the phosphorylation of tyrosyl residues in various proteins involved in the regulation of cell growth and differentiation (Wilks et al., Progress in Growth Factor Research 97 (1990) 2; Chan, A. C., and Shaw, A. S., Curr. Opin. Immunol. 8 (1996) 394-401). Such PTKs can be divided into receptor tyrosine kinases (e.g. EGFR/HER-1, c-erB2/HER-2, c-met, PDGFr, FGFr) and non-receptor tyrosine kinases (e.g. src, lck). It is known that many oncogenes encode proteins which are aberrant tyrosine kinases capable of causing cell transformation (Yarden, Y., and Ullrich, A., Annu. Rev. Biochem. 57 (1988) 443-478; Larsen et al., Ann. Reports in Med. Chem., 1989, Chpt. 13). Also over-expression of a normal proto-oncogenic tyrosine kinase may result in proliferative disorders.

It is known that receptor tyrosine kinases of the HER-family like HER-2 and EGFR (HER-1) are frequently aberrantly expressed in common human cancers such as breast cancer, gastrointestinal cancer (colon, rectal or stomach cancer), leukemia and ovarian, bronchial and pancreatic cancer. High levels of these receptors correlate with poor prognosis and response to treatment (Wright, C., et al., Br. J. Cancer 65 (1992) 118-121).

Accordingly, it has been recognized that inhibitors of receptor tyrosine kinases are useful as selective inhibitors of the growth of mammalian cancer cells. Therefore several small molecule compounds as well as monoclonal antibodies are in clinical trials for the treatment of various types of cancer (Baselga, J., and Hammond, L. A., Oncology 63 (Suppl. 1) (2002) 6-16; Ranson, M., and Sliwkowski, M. X., Oncology 63 (suppl. 1) (2002) 17-24).

Some substituted oxazoles are known in the art. WO 98/03505, EP 1 270 571, WO 01/77107, WO 03/031442 and WO 03/059907 disclose related heterocyclic compounds as tyrosine kinase inhibitors.

However there remains a need for new compounds with improved therapeutic properties, such as enhanced activity, decreased toxicity, better solubility and improved pharmacokinetic profile, to name only a few.

### SUMMARY OF THE INVENTION

The present invention relates to compounds of the general formula I, 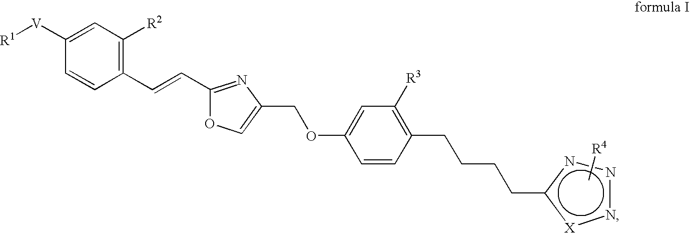
wherein
R¹ is halogenated alkyl;
R² is hydrogen or halogen;
R³ is hydrogen, alkyl or halogen;
R⁴ is hydrogen or alkyl; said alkyl being optionally substituted one or two times by —OH, —P(O)(alkyl)₂, —C(O)OH, —C(O)O-alkyl, —CN, morpholino, —S(O)-alkyl or —S(O)₂-alkyl;
V is —O— or —S—;
X is carbon or nitrogen; and
pharmaceutically acceptable salts thereof

The compounds of the present invention show activity as inhibitors of the HER-signalling pathway and therefore possess anti-proliferative activity.

Objects of the present invention are the compounds of formula I and their pharmaceutically acceptable salts, enantiomeric forms, diastereoisomers and racemates, the preparation of the above-mentioned compounds, medicaments containing them and their manufacture and pharmaceutical compositons of same, as well as the use of the above-mentioned compounds in the treatment, control or prevention of illnesses, especially of illnesses and disorders as mentioned above like common human cancers (e.g. breast cancer, gastrointestinal cancer (colon, rectal or stomach cancer), leukaemia and ovarian, bronchial and pancreatic cancer) or in the manufacture of corresponding medicaments.

### DETAILED DESCRIPTION OF THE INVENTION

The present invention relates to compounds of the general formula I, 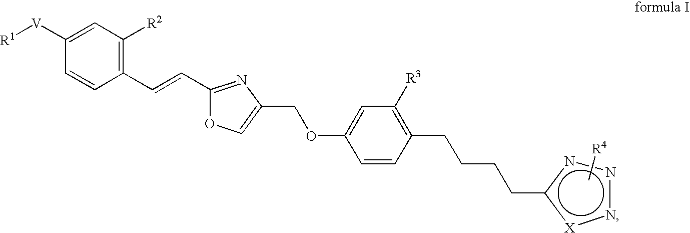
wherein
R¹ is halogenated alkyl;
R² is hydrogen or halogen;
R³ is hydrogen, alkyl or halogen;
R⁴ is hydrogen or alkyl, said alkyl being optionally substituted one or two times by —OH, —P(O)(alkyl)₂, —C(O)OH, —C(O)O-alkyl, —CN, morpholino, —S(O)-alkyl or —S(O)₂-alkyl;
V is —O— or —S—;
X is carbon or nitrogen; and
pharmaceutically acceptable salts and esters thereof.

The compounds of the present invention show activity as inhibitors of the HER-signalling pathway and therefore possess anti-proliferative activity. Objects of the present invention are the compounds of formula I and their pharmaceutically acceptable salts and esters, enantiomeric forms, diastereoisomers and racemates, the preparation of the above-mentioned compounds, medicaments containing them and their manufacture and pharmaceutical compositons of same, as well as the use of the above-mentioned compounds in the treatment, control or prevention of illnesses, especially of illnesses and disorders as mentioned above like common human cancers (e.g. breast cancer, gastrointestinal cancer (colon, rectal or stomach cancer), leukaemia and ovarian, bronchial and pancreatic cancer) or in the manufacture of corresponding medicaments.

As used herein, the term “alkyl” means a saturated, straight-chain or branched-chain hydrocarbon containing from 1 to 5, preferably 1 to 3, carbon atoms, such as methyl, ethyl, n-propyl, isopropyl, n-butyl, 2-butyl, t-butyl, n-pentyl, 3-methyl-butyl or 2-methyl-butyl.

As used herein, the term “halogenated alkyl” means an alkyl as defined above which is substituted with one or several halogen atoms, preferably fluorine or chlorine, especially fluorine. Examples are trifluoromethyl, 2,2,2-trifluoroethyl, perfluoroethyl and the like, preferably trifluoromethyl.

The term “halogen” as used herein means fluorine, chlorine, bromine and iodine, preferably fluorine, chlorine or bromine and more preferred fluorine and chlorine.

As used herein, when referring to the receptor tyrosine kinases of the HER-family like HER-2 and EGFR (HER-1), the acronym “HER” refers to human epidermal receptor and the acronym “EGFR” refers to epidermal growth factor receptor.

As used herein, in relation to mass spectrometry (MS) the term “ESI+” refers to positive electrospray ionization mode.

The term “pharmaceutically acceptable salt” is as used and defined on page 17.

The term “pharmaceutically acceptable ester” is as used and defined on page 18.

The term “therapeutically effective” or “therapeutically effective amount” as used herein means an amount of at least one compound of Fomula 1, or a pharmaceutically acceptable salt or ester thereof, that significantly inhibits proliferation and/or prevents differentiation of a human tumor cell, including human tumor cell lines.

Preferably R¹ in the definition of formula I represents trifluoromethyl.

The compounds of formula I can exist in different tautomeric forms and in variable mixtures thereof All tautomeric forms of the compounds of formula I and mixtures thereof are also an objective of the invention. For example, if X is nitrogen and R⁴ is hydrogen, the corresponding tetrazole ring of formula I can exist in two tautomeric forms as shown here below:

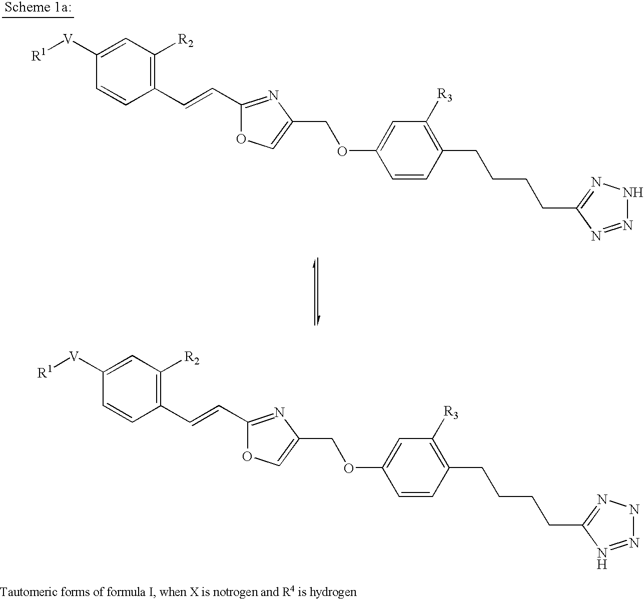

An embodiment of the invention are the compounds of formula I, wherein
R⁴ is hydrogen or alkyl; said alkyl being optionally substituted one or two times by —OH, —P(O)(alkyl)₂, —C(O)OH, —C(O)O-alkyl, —CN or morpholino.

Another embodiment of the invention are the compounds of formula I, wherein
R² is hydrogen; and
R³ is hydrogen or alkyl.

Another embodiment of the invention are the compounds of formula I, wherein
R² is hydrogen;
R³ is hydrogen; and
V is —O—.

Another embodiment of the invention are the compounds of formula I, wherein
R² is hydrogen;
R³ is hydrogen or alkyl; and
R⁴ is hydrogen or alkyl; said alkyl being optionally substituted one or two times by —OH, —P(O)(alkyl)₂, —C(O)OH, —C(O)O-alkyl, —CN or morpholino.

Another embodiment of the invention are the compounds of formula I, wherein
R² is hydrogen;
R³ is hydrogen;
R⁴ is hydrogen or alkyl; said alkyl being optionally substituted one or two times by —OH, —P(O)(alkyl)₂, —C(O)OH, —C(O)O-alkyl, —CN or morpholino; and
V is —O—.

Still another embodiment of the invention are the compounds of formula I, wherein
X is carbon.

Another embodiment of the invention are the compounds of formula I, wherein
R⁴ is hydrogen or alkyl; said alkyl being optionally substituted one or two times by —OH, —P(O)(alkyl)₂, —C(O)OH, —C(O)O-alkyl, —CN or morpholino; and
X is carbon.

Another embodiment of the invention are the compounds of formula I, wherein
R² is hydrogen;
R³ is hydrogen or alkyl; and
X is carbon.

Still another embodiment of the invention are the compounds of formula I, wherein
R² is hydrogen;
R³ is hydrogen;
V is —O—; and
X is carbon.

Another embodiment of the invention are the compounds of formula I, wherein
R² is hydrogen;
R³ is hydrogen or alkyl;
R⁴ is hydrogen or alkyl; said alkyl being optionally substituted one or two times by —OH, —P(O)(alkyl)₂, —C(O)OH, —C(O)O-alkyl, —CN or morpholino; and
X is carbon.

Still another embodiment of the invention are the compounds of formula I, wherein
R² is hydrogen;
R³ is hydrogen;
R⁴ is hydrogen or alkyl; said alkyl being optionally substituted one or two times by —OH, —P(O)(alkyl)₂, —C(O)OH, —C(O)O-alkyl, —CN or morpholino;
V is —O—; and
X is carbon.

Another embodiment of the invention are the compounds of formula I, wherein
R⁴ is hydrogen or alkyl; said alkyl being optionally substituted once by —OH— or —P(O)(alkyl)2; and
X is carbon.

Another embodiment of the invention are the compounds of formula I, wherein
R² is hydrogen; and
R³ is hydrogen or alkyl;
R⁴ is hydrogen or alkyl; said alkyl being optionally substituted once by —OH— or —P(O)(alkyl)2; and
X is carbon.

Still another embodiment of the invention are the compounds of formula I, wherein
R² is hydrogen;
R³ is hydrogen;
R⁴ is hydrogen or alkyl; said alkyl being optionally substituted once by —OH— or —P(O)(alkyl)₂;
V is —O—; and
X is carbon.

Such compounds are for example:
4-[4-(4-{2-[(E)-2-(4-Trifluoromethoxy-phenyl)-vinyl]-oxazol-4-ylmethoxy}-phenyl)-butyl]-1H-[1,2,3]triazole;
Methyl-5-[4-(4-{2-[(E)-2-(4-trifluoromethoxy-phenyl)-vinyl]-oxazol-4-ylmethoxy}-phenyl)-butyl]-1H-[1,2,3]triazole;
Methyl-4-[4-(4-{2-[(E)-2-(4-trifluoromethoxy-phenyl)-vinyl]-oxazol-4-ylmethoxy}-phenyl)-butyl]-2H-[1,2,3]triazole;
Methyl-4-[4-(4-{2-[(E) -2-(4-trifluoromethoxy-phenyl)-vinyl]-oxazol-4-ylmethoxy}-phenyl)-butyl]-1H-[1,2,3]triazole;
2-{4-[4-(4-{2-[(E)-2-(4-Trifluoromethoxy-phenyl)-vinyl]-oxazol-4-ylmethoxy}-phenyl)-butyl]-[1,2,3]triazol-2-yl}-ethanol;
2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethanol;
2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethanol;
3-{4-[4-(4-{2-[(E)-2-(4-Trifluoromethoxy-phenyl)-vinyl]-oxazol-4-ylmethoxy}-phenyl)-butyl]-[1,2,3]triazol-2-yl}-propan-1-ol;
3-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-propan-1-ol;
3-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-propan-1-ol;
1-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-1H-[1,2,3]triazole; and
2-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-2H-[1,2,3]triazole.

Still another embodiment of the invention are the compounds of formula I, wherein
R² is hydrogen;
R³ is alkyl;
R⁴ is hydrogen or alkyl; said alkyl being optionally substituted once by —OH— or —P(O)(alkyl)₂;
V is —O—; and
X is carbon.

Such compounds are for example:
4-[4-(2-Methyl-4-{2-[(E)-2-(4-Trifluoromethoxy-phenyl)-vinyl]-oxazol-4-ylmethoxy}-phenyl)-butyl]-1H-[1,2,3]triazole;
Methyl-4-[4-(2-methyl-4-{2-[(E)-2-(4-trifluoromethoxy-phenyl)-vinyl]-oxazol-4-ylmethoxy}-phenyl)-butyl]-2H-[1,2,3]triazole;
Methyl-4-[4-(2-methyl-4-{2-[(E)-2-(4-trifluoromethoxy-phenyl)-vinyl]-oxazol-4-ylmethoxy}-phenyl)-butyl]-1H-[1,2,3]triazole; and
Methyl-5-[4-(2-methyl-4-{2-[(E)-2-(4-trifluoromethoxy-phenyl)-vinyl]-oxazol-4-ylmethoxy}-phenyl)-butyl]-1H-[1,2,3]triazole.

Another embodiment of the invention are the compounds of formula I, wherein
R⁴ is alkyl; said alkyl being optionally substituted once by morpholino; and
X is carbon.

Another embodiment of the invention are the compounds of formula I, wherein
R² is hydrogen; and
R³ is hydrogen or alkyl;
R⁴ is alkyl; said alkyl being optionally substituted once by morpholino; and
X is carbon.

Still another embodiment of the invention are the compounds of formula I, wherein
R² is hydrogen;
R³ is hydrogen;
R⁴ is alkyl; said alkyl being optionally substituted once by morpholino; and
V is —O—; and
X is carbon.

Such compounds are for example:
4-(2-{4-[4-(4-{2-[(E)-2-(4-Trifluoromethoxy-phenyl)-vinyl]-oxazol-4-ylmethoxy}-phenyl)-butyl]-[1,2,3]triazol-2-yl}-ethyl)-morpholine;
4-(2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine; and
4-(2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine.

Another embodiment of the invention are the compounds of formula I, wherein
R⁴ is alkyl; said alkyl being substituted once by —C(O)OH, —C(O)O-alkyl or —CN; and
X is carbon.

Another embodiment of the invention are the compounds of formula I, wherein
R² is hydrogen; and
R³ is hydrogen or alkyl;
R⁴ is alkyl; said alkyl being substituted once by —C(O)OH, —C(O)O-alkyl or —CN; and
X is carbon.

Still another embodiment of the invention are the compounds of formula I, wherein
R² is hydrogen;
R³ is hydrogen;
R⁴ is alkyl; said alkyl being substituted once by —C(O)OH, —C(O)O-alkyl or —CN; and
V is —O—; and
X is carbon.

Such compounds are for example:
{4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetic acid methyl ester;
{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester;
{5-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester;
{4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetonitrile;
{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetonitrile;
{5-[4-(4-{2-[(E)-2-(4-Trifluoromethoxy-phenyl)-vinyl]-oxazol-4-ylmethoxy}-phenyl)-butyl]-[1,2,3 ]triazol-1-yl}-acetonitrile;
{4-[4-(4-{2-[2-(4-trifluoromethoxy-phenyl)-vinyl]-oxazol-4-ylmethoxy}-phenyl)-butyl]-[1,2,3]triazol-2-yl}-acetic acid sodium salt;
{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid sodium salt; and
{5-[4-(4-{2-[2-(4-Trifluoromethoxy-phenyl)-vinyl]-oxazol-4-ylmethoxy}-phenyl)-butyl]-[1,2,3]triazol-1-yl}-acetic acid sodium salt.

Another embodiment of the invention are the compounds of formula I, wherein X is nitrogen.

Another embodiment of the invention are the compounds of formula I, wherein
R⁴ is hydrogen or alkyl; said alkyl being optionally substituted one or two times by —OH, —P(O)(alkyl)₂, —C(O)OH, —C(O)O-alkyl, —CN or morpholino; and
X is nitrogen.

Another embodiment of the invention are the compounds of formula I, wherein
R² is hydrogen;
R³ is hydrogen or alkyl; and
X is nitrogen.

Still another embodiment of the invention are the compounds of formula I, wherein
R² is hydrogen;
R³ is hydrogen;
V is —O—; and
X is nitrogen.

Another embodiment of the invention are the compounds of formula I, wherein
R² is hydrogen;
R³ is hydrogen or alkyl;
R⁴ is hydrogen or alkyl; said alkyl being optionally substituted one or two times by —OH, —P(O)(alkyl)₂, —C(O)OH, —C(O)O-alkyl, —CN or morpholino; and
X is nitrogen.

Still another embodiment of the invention are the compounds of formula I, wherein
R² is hydrogen;
R³ is hydrogen;
R¹ is hydrogen or alkyl; said alkyl being optionally substituted one or two times by —OH, —P(O)(alkyl)₂, —C(O)OH, —C(O)O-alkyl, —CN or morpholino;
V is —O—; and
X is nitrogen.

Still another embodiment of the invention are the compounds of formula I, wherein
R⁴ is hydrogen or alkyl; said alkyl being optionally substituted once by —OH; and
X is nitrogen.

Another embodiment of the invention are the compounds of formula I, wherein
R² is hydrogen; and
R³ is hydrogen or alkyl;
R⁴ is hydrogen or alkyl; said alkyl being optionally substituted once by —OH; and
X is nitrogen.

Still another embodiment of the invention are the compounds of formula I, wherein
R² is hydrogen;
R³ is hydrogen;
R⁴ is hydrogen or alkyl; said alkyl being optionally substituted once by —OH; and
V is —O—; and
X is nitrogen.

Such compounds are for example:
5-[4-(4-{2-[(E)-2-(4-Trifluoromethoxy-phenyl)-vinyl]-oxazol-4-ylmethoxy}-phenyl)-butyl]-2H-tetrazole;
Methyl-5-[4-(4-{2-[(E)-2-(4-trifluoromethoxy-phenyl)-vinyl]-oxazol-4-ylmethoxy}-phenyl)-butyl]-1H-tetrazole;
Methyl-5-[4-(4-{2-[(E)-2-(4-trifluoromethoxy-phenyl)-vinyl]-oxazol-4-ylmethoxy}-phenyl)-butyl]-2H-tetrazole;
2-{5-[4-(4-{2-[(E)-2-(4-Trifluoromethoxy-phenyl)-vinyl]-oxazol-4-ylmethoxy}-phenyl)-butyl]-tetrazol-1-yl}-ethanol; and
2-{5-[4-(4-{2-[(E)-2-(4-Trifluoromethoxy-phenyl)-vinyl]-oxazol-4-ylmethoxy}-phenyl)-butyl]-tetrazol-2-yl}-ethanol.

Another embodiment of the invention are the compounds of formula I, wherein
R⁴ is alkyl; said alkyl being substituted once by —P(O)(alkyl)₂; and
X is nitrogen.

Another embodiment of the invention are the compounds of formula I, wherein
R² is hydrogen; and
R³ is hydrogen or alkyl;
R⁴ is alkyl; said alkyl being substituted once by —P(O)(alkyl)₂; and
X is nitrogen.

Still another embodiment of the invention are the compounds of formula I, wherein
R² is hydrogen;
R³ is hydrogen;
R⁴ is alkyl; said alkyl being substituted once by —P(O)(alkyl)₂; and
V is —O—; and
X is nitrogen.

Another embodiment of the invention are the compounds of formula I, wherein
R⁴ is alkyl; said alkyl being substituted once by —COOH, —C(O)O-alkyl or —CN; and
X is nitrogen.

Another embodiment of the invention are the compounds of formula I, wherein
R² is hydrogen; and
R³ is hydrogen or alkyl;
R⁴ is alkyl; said alkyl being substituted once by —COOH, —C(O)O-alkyl or —CN; and
X is nitrogen.

Still another embodiment of the invention are the compounds of formula I, wherein
R² is hydrogen;
R³ is hydrogen;
R⁴ is alkyl; said alkyl being substituted once by —COOH, —C(O)O-alkyl or —CN; and
V is —O—; and
X is nitrogen.

Another embodiment of the invention are the compounds of formula I, wherein
R⁴ is alkyl; said alkyl being substituted once by morpholino; and
X is nitrogen.

Another embodiment of the invention are the compounds of formula I, wherein
R² is hydrogen; and
R³ is hydrogen or alkyl;
R⁴ is alkyl; said alkyl being substituted once by morpholino; and
X is nitrogen.

Still another embodiment of the invention are the compounds of formula I, wherein
R² is hydrogen;
R³ is hydrogen;
R⁴ is alkyl; said alkyl being substituted once by morpholino; and
V is —O—; and
X is nitrogen.

Another embodiment of the invention are the compounds of formula I, wherein
R⁴ is alkyl; said alkyl being substituted one or two times by —P(O)(alkyl)₂.

An embodiment of the invention are the compounds of formula I, wherein
R² is hydrogen; and
R³ is hydrogen or alkyl; and
R⁴ is alkyl; said alkyl being substituted one or two times by —P(O)(alkyl)₂.

Another embodiment of the invention are the compounds of formula I, wherein
R² is hydrogen;
R³ is hydrogen; and
R⁴ is alkyl; said alkyl being substituted one or two times by —P(O)(alkyl)₂; and
V is —O—.

Still another embodiment of the invention is a process for the manufacture of the compounds of formula I, wherein

the compound of formula V 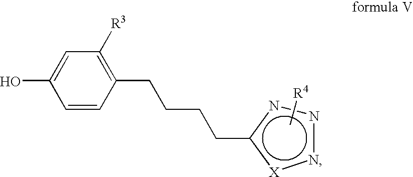
wherein R³ and R⁴ have the significance as given in formula I above or R⁴ is trityl, is reacted with a compound of formula VI 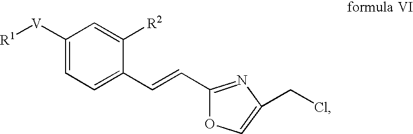
wherein R¹, R² and V have the significance given in formula I above, to give the respective compound of formula I;

said compound is isolated from the reaction mixture, and

if desired, converted into a pharmaceutically acceptable salt.

The derivatives of the general formula I or a pharmaceutically acceptable salt thereof, may be prepared by any process known to be applicable for the preparation of chemically-related compounds by the one skilled in the art. Such processes, when used to prepare the derivatives of formula I, or a pharmaceutically-acceptable salt thereof, are provided as a further feature of the invention and are illustrated by the following representative examples of scheme 1, in which, unless otherwise stated X, R¹, R², R³ and R⁴ have the significance given herein before. Necessary starting materials may be obtained by standard procedures of organic chemistry. The preparation of such starting materials is described within the accompanying examples or in WO 01/77107 and WO 03/059907 (herein incorporated by reference in its entirety). Alternatively necessary starting materials are obtainable by analogous procedures to those illustrated which are within the ordinary skill of an organic chemist.

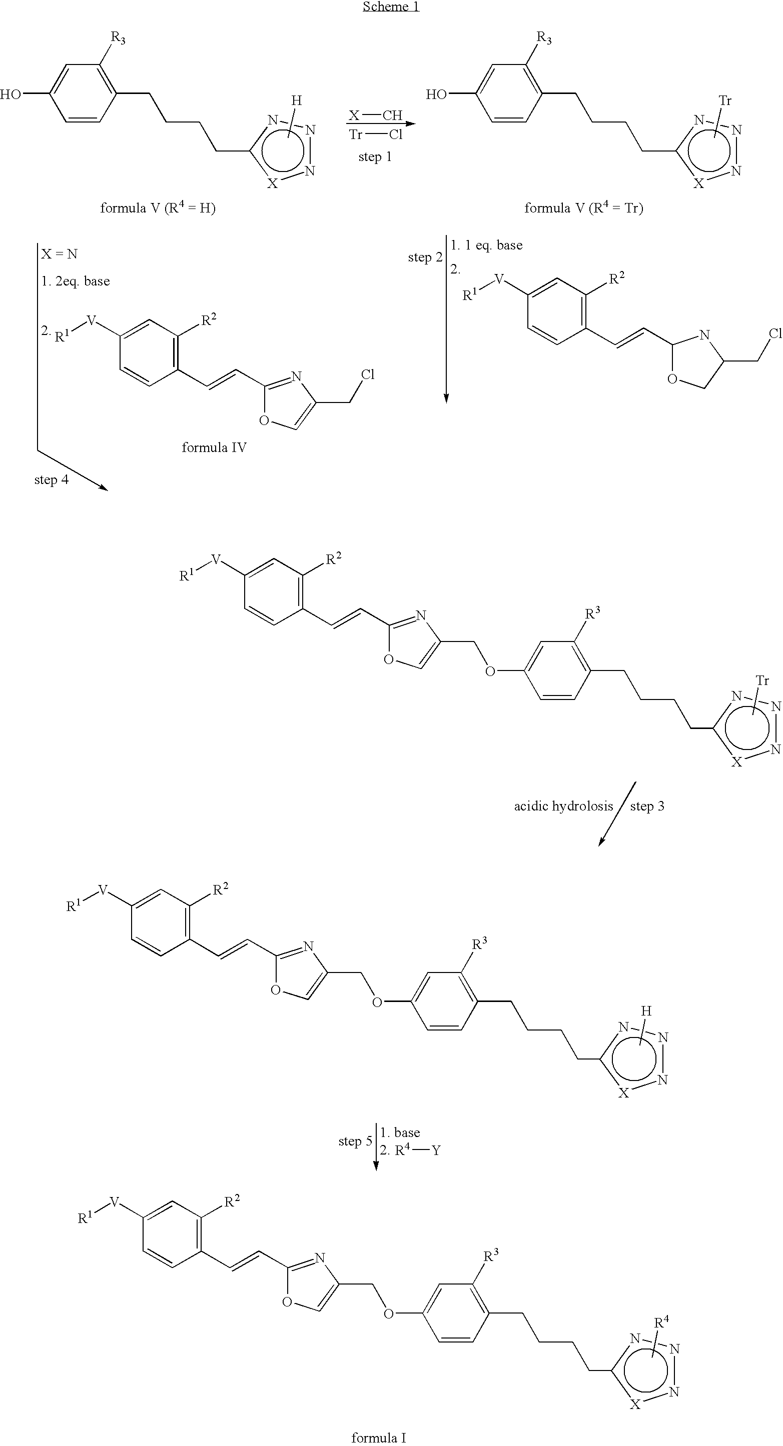

The compounds of the present invention are prepared in a straight forward manner (scheme 1). In case that formula V represents a triazole (X═CH), the triazole is first protected by a suitable protecting group (step 1). Most preferable for this process is the use of the trityl protecting group (Tr). The protected triazole is then reacted with a chloromethyloxazole of formula VI (step 2) in the presence of one equivalent of base. Suitable bases for this process are e.g. sodium hydride, Lithium Hexamethyldisilazide (LiHMDS), cesium carbonate, potassium carbonate or sodium hydroxide. Most preferable is sodium hydride. The obtained ethers are easily deprotected by heating with formic acid in THF (step 3).

In case that formula V represents a tetrazole (X═N), the phenol moiety of the tetrazole compound V is directly alkylated in the presence of 2 equivalents of a strong base with a chloromethyloxazole of formula VI (step 4). Suitable strong bases are for example sodium hydride or LiHMDS.

The obtained phenol ethers from both processes are finally alkylated to the products of formula I by a reagent R⁴—Y (step 5) in the presence of a base, where R⁴ has the meaning mentioned earlier and Y stand for a leaving group such as chlorine, bromine, iodine, mesylate or tosylate. Suitable bases for this process are sodium hydride, sodium hydroxide, LiHMDS, sodium carbonate, potassium carbonate or cesium carbonate.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g., Stahl, P. H., and Wermuth, G., (editors), Handbook of Pharmaceutical Salts, Verlag Helvetica Chimica Acta (VHCA), Zürich, (2002) or Bastin, R. J., et al., Organic Proc. Res. Dev. 4 (2000) 427-435, which are herein incorporated by reference in their entirety.

Preferred are the pharmaceutically acceptable salts, which are formed with p-toluenesulfonic acid, naphthalenesulfonic acid, naphthalenedisulfonic acid, methanesulfonic acid and hydrochloric acid.

The compounds according to the present invention may exist in the form of their pharmaceutically acceptable esters. The term “pharmaceutically acceptable ester” refers to a conventionally esterified compound of formula I having carboxyl group, which esters retain the biological effectiveness and properties of the compounds of formula I and are cleaved in vivo (in the organism) to the corresponding active carboxylic acid.

Information concerning esters and the use of esters for the delivery of pharmaceutical compounds is available in Design of Prodruge. Bundgaard H ed. (Elsevier, 1985). See also, H. Ansel et al., Pharmaceutical Dosage Forms and Drug Delivery Systems (6^{th} ed. 1995) at pp. 108-109; Krogsgaard-Larsen, et al., Textbook of Drug Design and Development (2d Ed. 19976) at pp. 152-191.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases which are commercially available.

The compounds of formula I and their pharmaceutically acceptable salts and esters possess valuable pharmacological properties. It has been found that said compounds inhibit the HER-signalling pathway and show anti-proliferative activity. Consequently the compounds of the present invention are useful in the therapy and/or prevention of illnesses with known over-expression of receptor tyrosine kinases of the HER-family like HER-2 and EGFR (HER-1), especially in the therapy and/or prevention of illnesses mentioned above. The activity of the present compounds as HER-signalling pathway inhibitors is demonstrated by the following exemplary biological assay:

CellTiter-Glo™ Assay in HEK293 Cells

The CellTiter-Glo™ Luminescent Cell Viability Assay (Promega) is a homogeneous method of determining the number of viable cells in culture based on quantitation of the ATP present, which signals the presence of metabolically active cells.

HEK293 cells (human embryonic kidney cell line transformed by Adenovirus 5 fragments, ATCC-No. CRL 1573) were cultivated in Dulbecco's Modified Eagle Medium (DMEM) with Glutamax™ (Invitrogen, 31966-021), 5% Fetal Calf Serum (FCS, Sigma Cat-No. F4135 (FBS)), 100 Units/ml penicillin/100 μg/ml streptomycin (=Pen/Strep from Invitrogen Cat. No. 15140)., For the assay the cells were seeded in 384 well plates, 5000 cells per well, in the same medium. The next day the test compounds were added in various concentrations ranging from 3 μM to 0.00015 μM (10 concentrations, 1:3 diluted). After 7 days the CellTiter-Glo™ assay was done according to the instructions of the manufacturer (CellTiter-Glo™ Luminescent Cell Viability Assay, from Promega). In brief: the cell-plate was equilibrated to room temperature for approximately 30 minutes and than the CellTiter-Glo™ reagent was added. The contents were carefully mixed for 15 minutes to induce cell lysis. After 45 minutes the luminescent signal was measured in Victor 2, (scanning multiwell spectrophotometer, Wallac).

Details:

1st Day:

Medium: Dulbecco's Modified Eagle Medium (DMEM) with Glutamax™ (Invitrogen, 31966-021), 5% Fetal Calf Serum (FCS, Sigma Cat-No. F4135 (FBS)), Pen/Strep (Invitrogen Cat. No. 15140).

HEK293 (ATCC-No. CRL 1573): 5000 cells in 60 μl per well of 384 well plate (Greiner 781098, white plates)

Incubate 24 h at 37° C., 5% CO₂

2^{nd} Day: Induction (Substance Testing):

In general the dilution steeps are 1:3
a) Add 8 μl of 10 mM stock solution of compound to 72 μl DMSO
b) dilute 9×1:3 (always 30 μl to 60 μl DMSO) in this DMSO dilution row (results in 10 wells with concentrations from 1000 μM to 0.06 μM)
c) dilute each concentration 1:4.8 (10 μl compound dilution to 38 μl medium)
d) dilute each concentration 1:10 (10 μl compound dilution to 90 μl medium)
e) add 10 μl of every concentration to 60 μl medium in the cell plate
resulting in final concentration of DMSO: 0.3% in every well
and resulting in final concentration of compounds from 3 μM to 0.00015 μM
Incubate 168 h (7 days) at 37° C., 5% CO₂

Analysis:
Add 30 μl CellTiter-Glo™ Reagent/well,
shake 15 minutes at room temperature
incubate further 45 minutes at room temperature without shaking.

Measurement:
Victor 2 scanning multiwell spectrophotometer (Wallac), Luminescence mode
Determine IC50 with XL-fit (XLfit software (ID Business Solution Ltd., Guilford, Surrey, UK)).

A significant inhibition of HEK293 cell viability was detected, which is exemplified by the compounds shown in Table 1.

TABLE 1
Results:

| Compound of Example(s) | IC50 HEK293 [nM] |
|---|---|
| 1.1 | 128 |
| 1.6 | 166 |
| 1.2, 1.4, 1.7, 1.18, 1.19, 2.2 | 5-250 |
| 1.14, 1.16, 1.17, 2.1, 2.4 | 250-1500 |

MTT Assay in A549 Cells:

To further show the activity of the compounds according to this invention, their effects on a human colon carcinoma cell line was evaluated using a standard MTT-assay. MTT (3-(4,5-dimethylthiazol-2-yl)-2,5-diphenyl tetrazolium bromide) is widely used for the quantitative determination of cytotoxic effects or in vitro chemosensitivity of tumor cells. The assay is based on the cleavage of the yellow tetrazolium salt (MTT) to purple formazan crystals by metabolic active cells. For details, see Rubinstein, L. V., et al., J. Natl. Cancer Inst. 82 (1990) 1113-1118.

A549 cells (human lung carcinoma cell line ATTC-No. CCL-185) were cultivated in RPMI (Roswell Park Memorial Institute) 1640 medium with GlutaMAX™ I (Invitrogen, Cat-No. 61870-010), 2,5% Fetal Calf Serum (FCS, Sigma Cat-No. F4135 (FBS)); 100 Units/ml penicillin/100 μg/ml streptomycin (=Pen/Strep from Invitrogen Cat. No. 15140). For the assay the cells were seeded in 384 well plates, 900 cells per well, in the same medium. The next day compounds (dissolved 10 mM in dimethylsulfoxide (DMSO)) were added in various concentrations ranging from 3 μM to 0.15 nM (10 concentrations, 1:3 diluted). After 5 days MTT assay was done mainly according to the instructions of the manufacturer (Cell proliferation kit I, MTT, from Roche Molecular Biochemicals). In brief: MTT labeling reagent (3-(4,5-dimethylthiazol-2-yl)-2,5-diphenyltetrazolium bromide (MTT)) was added to a final concentration of 0.5 mg/ml, added and incubated for 4 hrs at 37° C., 5% CO₂. During this incubation time purple formazan crystals are formed. After addition of the solubilization solution (20% Sodium Dodecyl Sulfate (SDS) in 0.02 M HCl) the plates were incubated overnight at 37° C., 5% CO₂. After careful mixing plates were measured in Victor 2 scanning multiwell spectrophotometer (Wallac) at 550 nm.

A decrease in number of living cells results in a decrease in the total metabolic activity in the sample. The decrease directly correlates to the amount of purple color resulting from the solubilization of the purple formazan crystals.

Details:

1^{st} Day: Cells:

A549: 900 cells in 60 μl per well of 384 well plate (Greiner)
Medium: RPMI 1640, 2.5% FCS, glutamine, Pen/Strep.
Incubate 1 day at 37° C.

Induction:
Dilution of compound in DMSO: 3 μl 10 mM+27 μl DMSO, dilute 1:3
Add 2 μl of compound dilution row to 95 μl of medium
Add 10 μl of compound dilution to 60 μl medium in test plate resulting in 0.3% DMSO per well
Incubate 120 h (5 days) at 37° C., 5% CO₂

Analysis:
Add 7 μl MTT (5 mg/7 ml/well), incubate 4 h at 37° C.
Add 30 μl lysis buffer (20% SDS, 0.04 N HCl) per well
Incubate overnight at 37° C.

Measurement:
Victor 2 scanning multiwell spectrophotometer (Wallac) at 550 nm

Determination of IC₅₀ was done using XL-fit (XLfit software (ID Business Solution Ltd., Guilford, Surrey, UK)).

TABLE 2
Results

| Compound of Example(s) | IC₅₀ A549 [nM] |
|---|---|
| 1-2 | 7 |
| 2-2 | 17 |
| 1.3, 1.4, 2.3, 2.5 | 5-200 |
| 1.1, 2.1, 2.4 | 200-1000 |

In vivo Assay on Tumor Inhibition:

To generate primary tumors, Non-Small-Cell Lung Cancer (NSCLC) (e.g. Calu-3 (ATTC HTB-55) or A549 (ATTC CCL-185)) cells (4-5.0×10⁶ in a volume of 100 μl) are injected subcutaneously into the left flank of female SCID beige (Severe Combined Immunodeficient/beige mice available from Charles River, Sulzfeld, Germany) or BALB/c nude (BALB/c Nude Spontaneous Mutant Mice (homozygotes) available from Taconic Europe, Ry, Denmark) mice. The cells are thawed and expanded in vitro before use in the experiment. Mice are assigned to the treatment groups 14-21 days after cell injection. For grouping (n=10-15 mice per group), the animals are randomized to get a similar mean primary tumor volume of ca. 100-150 mm³ per group. The test compounds are administered orally once per day as a suspension in 7.5% gelatine 0.22% NaCl with an administration volume of 10 ml/kg based on actual body weights. Treatment is initiated one day after staging, and carried out until day 20-50, the final day of the study. The subcutaneous primary tumors are measured twice weekly, starting prior to randomisation, in two dimensions (length and width) using an electronic caliper. The volume of the primary tumor is calculated using the formula: V[mm³]=(length [mm]×width [mm]×width [mm])/2. In addition, the body weight of all animals is recorded at least twice weekly. Finally, at the end of the study the tumors are explanted and weighed.

In one example of the above procedure, treatment was started on or about the 20^{th}-21^{st} day of the experiment (after cell injection) and the treatment was carried out for 23 days (about 44 days of experiment). In this example, primary tumor volume was calculated according to NCI protocol (TW=½ ab², where a and b were long and short diameters of tumor mass in mm). Calculation started at staging until study day 44 and values were documented as mean, median and SD. Tumor weight was determined by analytic scale at termination (day 44/45).

Median tumor growth inhibition for volume (T/C) was calculated according to the NCI formula: 1−([TV_{treated} (day 44-21)×100/TV_{control} (day 44-21)])×100. Then example 1.2 showed 50% Tumor Growth Inhibition (TGI) in A549-Cell-Xenograft at a daily dosage of 80 mg/kg after 23 days of treatment (44 days of experiment), and Example 3.2 showed 72% Tumor Growth inhibition (TGI) in A549-Cell-Xenograft at a daily dosage of 80 mg/kg after 23 days of treatment (44 days of experiment).

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically inert, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions comprise e.g. the following:

a) Tablet Formulation (Wet Granulation):

| Item | Ingredients | mg/tablet | | | |
|---|---|---|---|---|---|
| 1. | Compound of formula (I) | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Exemption Manufacturing Procedure:
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

b) Capsule Formulation:

| Item | Ingredients | mg/capsule | | | |
|---|---|---|---|---|---|
| 1. | Compound of formula (I) | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Exemption Manufacturing Procedure:
1. Mix items 1, 2 and 3 in a suitable mixer for 30 minutes.
2. Add items 4 and 5 and mix for 3 minutes.
3. Fill into a suitable capsule.
   c) Micro Suspension
1. Weigh 4.0 g glass beads in custom made tube GL 25, 4 cm (the beads fill half of the tube).
2. Add 50 mg compound, disperse with spatulum and vortex.
3. Add 2 ml gelatin solution (weight beads:gelatin solution=2:1) and vortex.
4. Cap and wrap in aluminium foil for light protection.
5. Prepare a counter balance for the mill.
6. Mill for 4 hours, 20/s in a Retsch mill (for some substances up to 24 hours at 30/s).
7. Extract suspension from beads with two layers of filter (100 μm) on a filter holder, coupled to a recipient vial by centrifugation at 400 g for 2 min.
8. Move extract to measuring cylinder.
9. Repeat washing with small volumes (here 1 ml steps) until final volume is reached or extract is clear.
10. Fill up to final volume with gelatin and homogenise.

The above described preparation yields micro-suspensions of the compounds of formula I with particle sizes between 1 and 10 μm. The suspensions are suitable for oral applications and can be used in the in vivo assay described above.

Medicaments containing a compound of the present invention or a pharmaceutically acceptable salt thereof and a therapeutically inert carrier are also an object of the present invention, as is a process for their production, which comprises bringing one or more compounds of the present invention and/or pharmaceutically acceptable salts and, if desired, one or more other therapeutically valuable substances into a galenical administration form together with one or more therapeutically inert carriers.

In accordance with the invention the compounds of the present invention as well as their pharmaceutically acceptable salts are useful in the control or prevention of illnesses. Based on their HER-signalling pathway inhibition and their antiproliferative activity, said compounds are useful for the treatment of diseases such as cancer in humans or animals and for the production of corresponding medicaments. The dosage depends on various factors such as manner of administration, species, age and/or individual state of health.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1000 mg, should be appropriate, although the upper limit may be exceeded when indicated. The daily dosage can be administered as a single dose or in divided doses, or for parenteral administration, it may be given as a continuous infusion.

Another embodiment of the invention is pharmaceutical composition, containing one or more compounds of formula I together with pharmaceutically acceptable excipients.

Still another embodiment of the invention is said pharmaceutical composition for the inhibition of tumor growth, specifically tumors of common human cancers and more preferably tumors of human colon carcinoma.

Still another embodiment of the invention is the use of a compound of formula I for the treatment of cancer, specifically common human cancers (e.g. breast cancer, gastrointestinal cancer (colon, rectal or stomach cancer), leukaemia and ovarian, bronchial and pancreatic cancer).

Still another embodiment of the invention is the use of a compound of formula I for the manufacture of corresponding medicaments for the inhibition of tumor growth.

The following examples and references are provided to aid the understanding of the present invention, the true scope of which is set forth in the appended claims. It is understood that modifications can be made in the procedures set forth without departing from the spirit of the invention.

### EXAMPLES

### Example 1.1

### 4-[4-(4-{2-[(E)-2-(4-Trifluoromethoxy-phenyl)-vinyl]-oxazol-4-ylmethoxy}-phenyl)-butyl]-1H-[1,2,3]triazole

### 1-(4-Bromo-butyl)-4-methoxy-benzene

After starting the Grignard reaction by adding 5.00 ml 4-bromoanisole to a mixture of 4.86 g (0.20 mol) magnesium turnings and 100 ml tetrahydrofuran (THF), 20.00 ml 4-bromoanisole (total: 25.0 ml (37.4 g; 0.20 mol) were added at a pace sufficient to maintain reflux temperature. The reaction mixture was heated to reflux for additional 3 hours (h), cooled to room temperature (r.t.) and dropped at 0° C. within 1 h to a stirred solution prepared by mixing 129.6 g (71.6 ml, 0.60 mol) 1,4-dibromo-butane in 200 ml THF with a freshly prepared solution of 0.17 g (4.0 mmol) LiCl and 0.267 g (2.0 mmol) Cu(II)Cl₂ in 20 ml THF. Stirring was continued for 12 h at r.t. followed by the addition of 100 ml of a 20% ammonium chloride solution and 200 ml ethyl acetate. The water phase was extracted twice with 50 ml ethyl acetate, all organic phases were combined, dried over sodium sulphate and evaporated. The resulting oil was fractionated by vacuum distillation. Yield: 27.7 g (57%), b.p. 112-115° C./0.15 mbar.

¹H-NMR(400 MHz, D₆-DMSO): δ=1.65(quintet, 2H, CH₂—CH₂-Ph), 1.77(quintet, 2H, CH₂—CH₂—Br), 2.53(t, 2H, CH₂-Ph), 3.53(t, 2H, CH₂—Br), 3.71(s, 3H, OCH₃), 6.84(d, 2H, 3-H/5-H), 7.10(d, 2H, 2-H/6-H).

### 1-(4-Iodo-butyl)-4-methoxy-benzene

A mixture consisting of 30.2 g (124 mmol) 1-(4-bromo-butyl)-4-methoxy-benzene, 19.2 g (128 mmol) sodium iodide and 508 ml acetone was heated to reflux temperature for 1 h. The resulting suspension was cooled to r.t. and the precipitated sodium bromide removed by filtration. The filtrate was stripped off the solvents by vacuum distillation and the residue distributed between water and diethyl ether. After drying of the organic phase over sodium sulphate, vacuum distillation gave 34.9 g (97%) of the title compound as slightly yellow coloured liquid.

MS: M=290.0 (ESI).

### [6-(4-Methoxy-phenyl)-hex-1-ynyl]-trimethyl-silane

12.4 ml (19.8 mmol) of 1.6 M butyl-lithium in n-hexane was added dropwise at −78° C. to a solution of 1.94 g (2.80 ml, 19.8 mmol) trimethylsilylacetylene and 2.39 ml (19.8 mmol) 1,3-Dimethyl-3,4,5,6-tetrahydro-2(1H)-pyrimidinone (DMPU) in 30 ml THF. After stirring for 1 h at −78° C. a solution of 28.7 g (9.89 mmol) 1-(4-Iodo-butyl)-4-methoxy-benzene in ml THF was added at −78° C. and stirring continued for 30 min. The reaction mixture was allowed to warm to r.t. overnight and then hydrolysed by a saturated ammonium chloride solution. The water phase was extracted with diethyl ether and the combined organic phases were dried over sodium sulphate. Removal of solvents in vacuo gave 3.20 g yellow liquid, which still contained solvent and was used without further purification.

MS: M=260.1 (ESI).

¹H-NMR(400 MHz, CDCl₃): δ=0.15(s, 9H, Si(CH₃)₃), 1.57(quintet, 2H, CH₂—CH₂—C≡C), 1.70(quintet, 2H, CH₂—CH₂—Ar), 2.19(t, 2H, CH₂—C≡C), 2.59(t, 2H, CH₂—Ar), 3.78(s, 3H, OCH₃), 6.81(d, 2H, 3′-/5′-H), 7.08(d, 2H, 2′-H/6′-H).

### 1-Hex-5-ynyl-4-methoxy-benzene

A mixture of 3.20 g (12.3 mmol) [6-(4-methoxy-phenyl)-hex-1-ynyl]-trimethyl-silane, 50 ml methanol and 12.3 ml (24.6 mmol) 2N NaOH was stirred for 2 h at r.t. After neutralization with 13 ml 2N HCl methanol was distilled off and the aqueous phase extracted with diethyl ether. Drying (Na₂SO₄) and removal of solvents in vacuo gave 1.80 g (78%) of the title compound.

MS: M=188.1 (ESI).

¹H-NMR(400 MHz, CDCl₃): δ=1.55(quintet, 2H, CH₂—CH₂—C≡CH), 1.69(quintet, 2H, CH₂—CH₂—Ar), 1.93(s, 1H, ≡CH), 2.26(t, 2H, CH₂—C≡CH), 2.60(t, 2H, CH₂—Ar), 3.78(s, 3H, OCH₃), 6.83(d, 2H, 3′-/5′-H), 7.09(d, 2H, 2′-H/6′-H).

### 4-(4-(4-Methoxy-phenyl)-butyl)-1H-[1,2,3]triazole

A mixture of 1.80 g (9.56 mmol) 1-hex-5-ynyl-4-methoxy-benzene, 1.86 g (28.6 mmol) sodium azide, 1.53 g (28.6 mmol) ammonium chloride and 80 ml N,N-dimethylformamide (DMF) was kept at 125° C. for 7 d with an extra addition of 1.80 g sodium azide and 1.53 g ammonium chloride every day. After cooling to r.t. the dark reaction mixture was distributed between water and ethyl acetate. The organic phase was dried over sodium sulphate and the solvent distilled off. The residue was separated by HPLC on a RP18-endcapped column (methanol/water) to yield 450 mg 5-(4-(4-Methoxy-phenyl)-butyl)-2H-tetrazole and 500 mg 4-(4-(4-Methoxy-phenyl)-butyl)-1H-[1,2,3]triazole

### 5-(4-(4-Methoxy-phenyl)-butyl)-2H-tetrazole:

MS: M=233.3(AP+), 231.3(AP−).

¹H-NMR(400 MHz, CDCl₃): δ=1.67(quintet, 2H, CH₂—CH₂—Ar), 1.87(quintet, 2H, CH₂—CH₂-tetrazole), 2.56(t, 2H, CH₂—Ar), 3.08(t, 2H, CH₂-tetrazole), 3.74(s, 3H, OCH₃), 6.67(d, 2H, 3′-/5′-H), 6.97(d, 2H, 2′-/6′-H), 11.5-12.5(br, 1H, NH).

### 4-(4-(4-Methoxy-phenyl)-butyl)-1H-[1,2,3]triazole:

MS: M=232.2(APCI+), 230.2(APCI−).

¹H-NMR(400 MHz, D₆-DMSO): δ=1.50-1.65(m, 4H), 2.53(t, 2H, CH₂—Ar), 2.65(t, 2H, CH₂-triazole), 3.71(s, 3H, OCH₃), 6.83(d, 2H, 3′-/5′-H), 7.08(d, 2H, 2′-/6′-H), 7.5(br, 1H, 5-H-triazole), 14-15(br, 1H, NH).

### 4-(4-1H-[1,2,3]triazol-4-yl-butyl)-phenol

A mixture of 500 mg 4-(4-(4-methoxy-phenyl)-butyl)-1H-[1,2,3]triazole and 1.5 ml 48% hydrobromic acid was stirred at 80° C. for 9 h. After adjustment to pH=6 by addition of conc. sodium hydroxide solution, the aqueous layer was discarded and the remaining sticky residue purified by HPLC-MS(RP18, methanol/water 7:3, pH=2.3). Yield 170 mg (36%).

MS: M=218.2(APCI+), 216.2(APCI−).

¹H-NMR(400MHz, D₆-DMSO): δ=1.55(mc, 4H, CH₂), 2.48(t, 2H, CH₂—Ar), 2.64(t, 2H, CH₂-triazole), 6.65(d, 2H, 2′-/6′-H), 6.95(d, 2H, 3′-/5′-H), 7.58(br, 1H, 5-H-triazole), 9.08(br, 1H, NH).

### 4-[4-(1-Trityl-1H-[1,2,3]triazol-4-yl)-butyl]-phenol

A solution of 706 mg (5.06 mmol) triphenylchloromethane in 5.0 ml DMF was added at 0° C. to a solution of 500 mg (2.30 mmol) 4-(4-1H-[1,2,3]triazol-4-yl-butyl)-phenol and 512 mg (5.06 mmol) triethylamine in 5.0 ml DMF. The mixture was allowed to reach r.t. overnight and solvents were removed in vacuo. After distribution of the residue between water and ethyl acetate, the organic phase was dried (sodium sulphate), solvents distilled off and the residue purified by column chromatography on silica gel (heptane/ethyl acetate 2:1). Yield 610 mg (58%).

MS: M=460.2(ESI+), 482.2 (ESI+, M+Na⁺), 458.2 (ESI−).

¹H-NMR(400 MHz, CDCl₃): δ=1.59(mc, 2H, CH₂—CH₂—Ar), 1.67(mc, 2H, CH₂—CH₂-triazole), 2.53(t, 2H, CH₂—Ar), 2.71(t, 2H, CH₂-triazole), 5.10(s, 1H, OH), 6.72(d, 2H, 2′-/6′-H), 6.97(d, 2H, 3′-/5′-H), 7.05-7.40(m, 15H, trityl).

### 4-[4-(4-{2-[(E)-2-(4-Trifluoromethoxy-phenyl)-vinyl]-oxazol-4-ylmethoxy}-phenyl)-butyl]-1-trityl-1H-[1,2,3]triazole

34.5 mg (1.33 mmol) 95% sodium hydride were given at 0° C. to a solution of 610 mg 4-[4-(1-trityl-1H-[1,2,3]triazol-4-yl)-butyl]-phenol in 7.0 ml N,N-dimethylformamide and stirred for 10 min. 403 mg (1.33 mmol) 4-chloromethyl-2-[2-(4-trifluoromethoxy-phenyl)-vinyl]-oxazole in 3.0 ml DMF were added and stirring continued overnight at 25° C. The reaction mixture was poured into water, after standing for 2 h the precipitate was isolated by filtration, washed with water and dried to yield 620 mg (64%) of the title compound

MS: M=485.3(APCI+, M-trityl), 483.3(APCI−).

### 4-[4-(4-{2-[(E)-2-(4-Trifluoromethoxy-phenyl)-vinyl]-oxazol-4-ylmethoxy}-phenyl)-butyl]-1H-[1,2,3]triazole

A mixture of 10.4 g (14.3 mmol) 4-[4-(4-{2-[(E)-2-(4-Trifluoromethoxy-phenyl)-vinyl]-oxazol-4-ylmethoxy}-phenyl)-butyl]-1-trityl-1H-[1,2,3]triazole, 11.8 g formic acid and 100 ml THF was stirred at 60° C. for 36 h. The reaction mixture was added to water, neutralized with 2 N sodium hydroxide to pH=6 and extracted with ethyl acetate. The organic phase was dried (Na₂SO₄), solvents distilled off in vacuo and the residue treated with ethyl acetate, filtered and washed with ether. 3.47 g (41%) of example 1.1

MS: M=485.2(APCI+), 483.3(APCI−).

¹H-NMR(400 MHz, D₆-DMSO): δ=1.58(mc, 4H), 2.54(t, 2H, —CH₂Ph), 2.65(t, 2H, —CH₂-triazole), 4.97(s, 2H, —OCH₂—), 6.94(d, 2H, 3′-/5′-H), 7.11(d, 2H, 2′-/6′-H), 7.21(d, 1H, ═CH), 7.40(d, 2H, 3″-/5″-H, ArOCF₃), 7.56(d, 1H, ═CH), 7.87(d, 2H, 2″-/6″-H, ArOCF₃), 8.20(s, 1H, oxazole), 14.5(br, 1H, NH).

### Example 1.2

### Methyl-5-[4-(4-{2-[(E)-2-(4-trifluoromethoxy-phenyl)-vinyl]-oxazol-4-ylmethoxy}-phenyl)-butyl]-1H-[1,2,3]triazole

The title compound and 2-Methyl-4-[4-(4-{2-[(E)-2-(4-trifluoromethoxy-phenyl)-vinyl]-oxazol-4-ylmethoxy}-phenyl)-butyl]-2H-[1,2,3]triazole (example 1.3) and 1-Methyl-4-[4-(4-{2-[(E)-2-(4-trifluoromethoxy-phenyl)-vinyl]-oxazol-4-ylmethoxy}-phenyl)-butyl]-1H-[1,2,3]triazole (example 1.4) were prepared from 4-[4-(4-{2-[(E)-2-(4-Trifluoromethoxy-phenyl)-vinyl]-oxazol-4-ylmethoxy}-phenyl)-butyl]-1H-[1,2,3]triazole and iodomethane as described in for the corresponding methyl derivative in example 3.2.

MS: M=498.1(EI), 499.1(ESI+)

¹H-NMR(500 MHz, D₆-DMSO): δ=1.59(m, 4H, CH₂—CH₂—Ar, CH₂—CH₂-triazole), 2.56(t, 2H, CH₂—Ar), 2.67(t, 2H, CH₂-triazole), 3.90(s, 3H, NCH₃), 4.98(s, 2H, OCH₂), 6.95(d, 2H, 3′-/5′-H), 7.12(d, 2H, 2′-/6′-H), 7.21(d, J=16.4 Hz, 1H, vinyl-H), 7.40(d, 2H, ArOCF₃), 7.46(s, 1H, triazole), 7.57(d, J=16.4 Hz, 1H, vinyl-H), 7.87(d, 2H, ArOCF₃), 8.21(s, 1H, oxazole).

### Example 1.3

### Methyl-4-[4-(4-{2-[(E)-2-(4-trifluoromethoxy-phenyl)-vinyl]-oxazol-4-ylmethoxy}-phenyl)-butyl]-2H-[1,2,3]triazole

MS: M=498.1(EI), 499.1(ESI+)

¹H-NMR(500 MHz, D₆-DMSO): δ=1.57(m, 4H, CH₂—CH₂—Ar, CH₂—CH₂-triazole), 2.54(t, 2H, CH₂—Ar), 2.61(t, 2H, CH₂-triazole), 4.05(s, 3H, NCH₃), 4.98(s, 2H, OCH₂), 6.94(d, 2H, 2′-/6′-H), 7.11(d, 2H, 3′-/5′-H), 7.20(d, J=16.4 Hz, 1H, vinyl-H), 7.40(d, 2H, ArOCF₃), 7.48(s, 1H, triazole), 7.56(d, J=16.4 Hz, 1H, vinyl-H), 7.86(d, 2H, ArOCF₃), 8.20(s, 1H, oxazole).

### Example 1.4

### Methyl-4-[4-(4-{2-[(E)-2-(4-trifluoromethoxy-phenyl)-vinyl]-oxazol-4-ylmethoxy}-phenyl)-butyl]-1H-[1,2,3]triazole

MS: M=498.1(EI), 499.1(ESI+)

¹H-NMR(500 MHz, D₆-DMSO): δ=1.57(m, 4H, CH₂—CH₂—Ar, CH₂—CH₂-triazole), 2.54(t, 2H, CH₂—Ar), 2.61(t, 2H, CH₂-triazole), 3.98(s, 3H, NCH₃), 4.98(s, 2H, OCH₂), 6.94(d, 2H, 2′-/6′-H), 7.11(d, 2H, 3′-/5′-H), 7.20(d, J=16.4 Hz, 1H, vinyl-H), 7.40(d, 2H, ArOCF₃), 7.56(d, J=16.4 Hz, 1H, vinyl-H), 7.75(s, 1H, triazole), 7.86(d, 2H, ArOCF₃), 8.20(s, 1H, oxazole).

### Example 1.5

### 2-{4-[4-(4-{2-[(E)-2-(4-Trifluoromethoxy-phenyl)-vinyl]-oxazol-4-ylmethoxy}-phenyl)-butyl]-[1,2,3]triazol-2-yl}-ethanol

17.2 mg (0.68 mmol) 95% sodium hydride were added to a solution of 250 mg (0.516 mmol) 4-[4-(4-{2-[(E)-2-(4-Trifluoromethoxy-phenyl)-vinyl]-oxazol-4-ylmethoxy}-phenyl]-butyl]-1H-[1,2,3]triazole in 4.0 ml DMF and stirred for 60 min. (precipitation of sodium salt). 85 mg (0.68 mmol) 2-bromoethanol were added, the mixture stirred overnight to give a clear solution and evaporated. The remaining mixture was given to water and extracted with ethyl acetate. The organic phase was dried (Na₂SO₄) and evaporated. Separation of the residue by HPLC/MS gave 110 mg of the title compound and 30 mg that were further purified on a OJ-chiral column (methanol/water 95:5) to yield 9 mg 2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethanol (example 1.6) and 6 mg 2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethanol (example 1.7).

¹H-NMR(500 MHz, CDCl₃): δ=1.72(m, 4H, CH₂—CH₂—Ar, CH₂—CH₂-triazole), 2.62(t, 2H, CH₂—Ar), 2.71(t, 2H, CH₂-triazole), 4.09(t, 2H, CH₂OH), 4.52(t, 2H, CH₂N), 5.04(s, 2H, OCH₂), 6.93(m, 3H, 2′-/6′-H, vinyl-H), 7.12(d, 2H, 3′-/5′-H), 7.28(d, 2H, ArOCF₃), 7.38(s, 1H, triazole), 7.51(d, J=16.4 Hz, 1H, vinyl-H), 7.56(d, 2H, ArOCF₃), 7.68(s, 1H, oxazole).

### Example 1.6

### 2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethanol

¹H-NMR(500 MHz, CDCl₃): δ=1.70(m, 4H, CH₂—CH₂—Ar, CH₂—CH₂-triazole), 2.59(t, 2H, CH₂—Ar), 2.71(t, 2H, CH₂-triazole), 4.05(t, 2H, CH₂OH), 4.42(t, 2H, CH₂N), 5.00(s, 2H, OCH₂), 6.89(m, 3H, 2′-/6′-H, vinyl-H), 7.11(d, 2H, 3′-/5′-H), 7.23(d, 2H, ArOCF₃), 7.31(s, 1H, triazole), 7.51(d, J=16.7 Hz, 1H, vinyl-H), 7.54(d, 2H, ArOCF₃), 7.66(s, 1H, oxazole).

### Example 1.7

### 2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethanol

¹H-NMR(500 MHz, CDCl₃) δ=1.68(m, 4H, CH₂—CH₂—Ar, CH₂—CH₂-triazole), 2.61(t, 2H, CH₂—Ar), 2.67(t, 2H, CH₂-triazole), 4.10(t, 2H, CH₂OH), 4.27(t, 2H, CH₂N), 5.01(s, 2H, OCH₂), 6.91(d, J=16.7 Hz), 6.93(d, 2H, 2′-/6′-H), 7.08(d, 2H, 3′-/5′-H), 7.24(d, 2H, ArOCF₃), 7.41(s, 1H, triazole), 7.51(d, J=16.7 Hz, 1H, vinyl-H), 7.56(d, 2H, ArOCF₃), 7.68(s, 1H, oxazole).

### Example 1.8

### {4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetic acid methyl ester

13.6 mg (0.537 mmol) 95% sodium hydride were added to a solution of 200 mg (0.413 mmol) 4-[4-(4-{2-[(E)-2-(4-Trifluoromethoxy-phenyl)-vinyl]-oxazol-4-ylmethoxy}-phenyl)-butyl]-1H-[1,2,3]triazole in 4.0 ml DMF and stirred for 60 min. (precipitation of sodium salt). 79 mg (0.52 mmol) methyl 2-bromoacetate were added, the mixture stirred overnight to give a clear solution and evaporated. The remaining mixture was given to water and extracted with ethyl acetate. The organic phase was dried (Na₂SO₄) and evaporated. Separation of the residue by HPLC/MS gave 40 mg of the title compound and 33 mg that were further purified on a OJ-chiral column (methanol) to yield 10 mg {4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester (example 1.9) and 5 mg {5-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester (example 1.10).

MS: 557.24(ESI+)

¹H-NMR(500 MHz, CDCl₃): δ=1.68(m, 4H, CH₂—CH₂—Ar, CH₂—CH₂-triazole), 2.56(t, 2H, CH₂—Ar), 2.70(t, 2H, CH₂-triazole), 3.78(s, 3H, OCH₃), 5.01(s, 2H, OCH₂), 5.17(d, 2H, NCH₂), 6.90(m, 3H, 3′-/5′-H, vinyl-H), 7.09(d, 2H, 2′-/6′-H), 7.23(d, 2H, ArOCF₃), 7.42(s, 1H, triazole), 7.51(d, J=16.4 Hz, 1H, vinyl-H), 7.54(d, 2H, ArOCF₃), 7.67(s, 1H, oxazole).

### Example 1.9

### {4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester

MS: 557.24(ESI+)

¹H-NMR(500 MHz, CDCl₃); δ=1.68(m, 4H, CH₂—CH₂—Ar, CH₂—CH₂-triazole), 2.60(t, 2H, CH₂—Ar), 2.76(t, 2H, CH₂-triazole), 3.80(s, 3H, OCH₃), 5.01(s, 2H, OCH₂), 5.13(d, 2H, NCH₂), 6.91(m, 3H, 3′-/5′-H, vinyl-H), 7.10(d, 2H, 2′-/6′-H), 7.25(d, 2H, ArOCF₃), 7.38(s, 1H, triazole), 7.51(d, J=16.7 Hz, 1H, vinyl-H), 7.55(d, 2H, ArOCF₃), 7.66(s, 1H, oxazole).

### Example 1.10

### {5-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester

MS: 557.24(ESI+)

¹H-NMR(500 MHz, CDCl₃): δ=1.68(m, 4H, CH₂—CH₂—Ar, CH₂—CH₂-triazole), 2.60(m, 4H, CH₂—Ar, CH₂-triazole), 3.78(s, 3H, OCH₃), 5.02(s, 2H, OCH₂), 5.05(d, 2H, NCH₂), 6.93(m, 3H, 3′-/5′-H, vinyl-H), 7.09(d, 2H, 2′-/6′-H), 7.24(d, 2H, ArOCF₃), 7.46(s, 1H, triazole), 7.51(d, J=16.7 Hz, 1H, vinyl-H), 7.55(d, 2H, ArOCF₃), 7.66(s, 1H, oxazole).

### Example 1.11

### {4-[4-(4-{2-[2-(4-trifluoromethoxy-phenyl)-vinyl]-oxazol-4-ylmethoxy}-phenyl)-butyl]-[1,2,3]triazol-2-yl}-acetic acid sodium salt

180 μ1 (0.359 mmol) of 2 N NaOH were added to a solution of 100 mg (0.180 mmol) {4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetic acid methyl ester in 3.0 ml THF and 3.0 ml methanol and stirred for 10 min. The resulting precipitate was collected, washed with methanol and dried. Yield: 50 mg 1.11.

¹H-NMR(500 MHz, D₆DMSO): δ=1.59(m, 4H, CH₂—CH₂—Ar, CH₂—CH₂-triazole), 2.53(t, 2H, CH₂—Ar), 2.59(t, 2H, CH₂-triazole), 4.55(d, 2H, NCH₂), 4.98(s, 2H, OCH₂), 6.94(d, 2H, 3′-/5′-H),), 7.12(d, 2H, 2′-/6′-H), 7.21(d, J=16.7 Hz, vinyl-H), 7.35(s, 1H, triazole), 7.40(d, 2H, ArOCF₃), 7.57(d, J=16.7 Hz, 1H, vinyl-H), 7.87(d, 2H, ArOCF₃), 8.21(s, 1H, oxazole).

### Example 1.12

### {4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid

¹H-NMR(500 MHz, D₆DMSO): δ=1.60(m, 4H, CH₂—CH₂—Ar, CH₂—CH₂-triazole), 2.56(t, 2H, CH₂—Ar), 2.65(t, 2H, CH₂-triazole), 4.99(s, 2H, OCH₂), 5.19(d, 2H, NCH₂), 6.94(d, 2H, 3′-/5′-H), 7.12(d, 2H, 2′-/6′-H), 7.21(d, J=16.7 Hz, vinyl-H), 7.40(d, 2H, ArOCF₃), 7.56(d, J=16.7 Hz, 1H, vinyl-H), 7.80(s, 1H, triazole), 7.87(d, 2H, ArOCF₃), 8.20(s, 1H, oxazole).

### Example 1.13

### {5-[4-(4-{2-[2-(4-Trifluoromethoxy-phenyl)-vinyl]-oxazol-4-ylmethoxy}-phenyl)-butyl]-[1,2,3]triazol-1-yl}-acetic acid sodium salt

¹H-NMR(500 MHz, D₆DMSO): δ=1.60(m, 4H, CH₂—CH₂—Ar, CH₂—CH₂-triazole), 2.55(t, 2H, CH₂—Ar), 2.58(t, 2H, CH₂-triazole), 4.50(d, 2H, NCH₂), 4.99(s, 2H, OCH₂), 6.95(d, 2H, 3′-/5′-H), 7.14(d, 2H, 2′-/6′-H), 7.21(d, J=16.7 Hz, vinyl-H), 7.33(s, 1H, triazole), 7.40(d, 2H, ArOCF₃), 7.57(d, J=16.7 Hz, 1H, vinyl-H), 7.87(d, 2H, ArOCF₃), 8.20(s, 1H, oxazole).

### Example 1.14

### {4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetonitrile

13.6 mg (0.537 mmol) 95% sodium hydride were added to a solution of 200 mg (0.413 mmol) 4-[4-(4-{2-[(E)-2-(4-Trifluoromethoxy-phenyl)-vinyl]-oxazol-4-ylmethoxy}-phenyl)-butyl]-1H-[1,2,3]triazole in 4.0 ml DMF and stirred for 60 min. (precipitation of sodium salt). 85 mg (0.68 mmol) 2-iodoacetonitrile were added, the mixture stirred overnight to give a clear solution and evaporated. The remaining mixture was given to water and extracted with ethyl acetate. The organic phase was dried (Na₂SO₄) and evaporated. Separation of the residue by HPLC/MS gave 70 mg of the title compound and 46 mg that were further purified on a OJ-chiral column (methanol) to yield 21 mg 2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetonitrile (example 1.15) and 19 mg 2-{5-[4-(4-{2-[(E)-2-(4-Trifluoromethoxy-phenyl)-vinyl]-oxazol-4-ylmethyl}-phenyl)-butyl]-[1,2,3]triazol-1-yl}-acetonitrile (example 1.16).

MS: 524.19(ESI+)

¹H-NMR(500 MHz, CDCl₃): δ=1.66(m, 4H, CH₂—CH₂—Ar, CH₂—CH₂-triazole), 2.57(t, 2H, CH₂—Ar), 2.70(t, 2H, CH₂-triazole), 5.02(s, 2H, OCH₂), 5.28(d, 2H, NCH₂), 6.89(d, J=16.4 Hz), 6.90(d, 2H, 3′-/5′-H), 7.09(d, 2H, 2′-/6′-H), 7.22(d, 2H, ArOCF₃), 7.44(s, 1H, triazole), 7.52(d, J=16.4 Hz, 1H, vinyl-H), 7.53(d, 2H, ArOCF₃), 7.67(s, 1H, oxazole).

### Example 1.15

### {4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetonitrile

MS: (ESI+)

¹H-NMR(500 MHz, CDCl₃): δ=1.66(m, 4H, CH₂—CH₂—Ar, CH₂—CH₂-triazole), 2.60(t, 2H, CH₂—Ar), 2.76(t, 2H, CH₂-triazole), 5.01(s, 2H, OCH₂), 5.29(d, 2H, NCH₂), 6.91(m, 3H, 3′-/5′-H), 7.09(d, 2H, 2′-/6′-H), 7.23(d, 2H, ArOCF₃), 7.44(s, 1H, triazole), 7.50(d, J=16.7 Hz, 1H, vinyl-H), 7.55(d, 2H, ArOCF₃), 7.66(s, 1H, oxazole).

### Example 1.16

### {5-[4-(4-{2-[(E)-2-(4-Trifluoromethoxy-phenyl)-vinyl]-oxazol-4-ylmethoxy}-phenyl)-butyl]-[1,2,3]triazol-1-yl}-acetonitrile

MS: (ESI+)

¹H-NMR(500 MHz, CDCl₃): δ=1.70(m, 4H, CH₂—CH₂—Ar, CH₂—CH₂-triazole), 2.64(t, 2H, CH₂—Ar), 2.74(t, 2H, CH₂-triazole), 5.02(s, 2H, OCH₂), 5.19(d, 2H, NCH₂), 6.91(d, 1H, J=16.7 Hz, vinyl-H), 6.94(d, 2H, 3′-/5′-H), 7.10(d, 2H, 2′-/6′-H), 7.23(d, 2H, ArOCF₃), 7.48(s, 1H, triazole), 7.53(d, J=16.7 Hz, 1H, vinyl-H), 7.55(d, 2H, ArOCF₃), 7.66(s, 1H, oxazole).

### Example 1.17

### 1-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-1H-[1,2,3]triazole

13.9 mg (0.55 mmol) 95% sodium hydride were added to a solution of 200 mg (0.413 mmol) 4-[4-(4-{2-[(E)-2-(4-Trifluoromethoxy-phenyl)-vinyl]-oxazol-4-ylmethoxy}-phenyl)-butyl]-1H-[1,2,3]triazole in 3.0 ml DMF and stirred for 60 min. (precipitation of sodium salt). 67.9 mg (0.537 mmol) chloromethyldimethylphosphanoxide were added, the mixture stirred overnight to give a clear solution and evaporated. The remaining mixture was given to water and extracted with ethyl acetate. The organic phase was dried (Na2SO4) and evaporated. Separation of the residue by HPLC/MS on a OJ-chiral column gave 80 mg of the title compound and 25 mg of 2-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-2H-[1,2,3]triazole (example 1.18).

MS: 575.4(ESI+)

¹H-NMR(500 MHz, CDCl₃): δ=1.65(m, 10H, CH₂—CH₂—Ar, CH₂—CH₂-triazole, CH₃), 2.57(t, 2H, CH₂—Ar), 2.78(t, 2H, CH₂-triazole), 5.00(s, 2H, OCH₂), 5.09(d, 2H, NCH₂), 6.92(d, 2H, 3′-/5′-H), 6.95(d, J=16.7 Hz, 1H, vinyl-H), 7.08(d, 2H, 2′-/6′-H), 7.24(d, 2H, ArOCF₃), 7.5(m, 3H, vinyl-H, ArOCF₃), 7.68(s, 1H, oxazole), 8.00(br, 1H, triazole).

### Example 1.18

### 2-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-2H-[1,2,3]triazole

13.9 mg (0.55 mmol) 95% sodium hydride were added to a solution of 200 mg (0.413 mmol) 4-[4-(4-{2-[(E)-2-(4-Trifluoromethoxy-phenyl)-vinyl]-oxazol-4-ylmethoxy}-phenyl)-butyl]-1H-[1,2,3]triazole in 3.0 ml DMF and stirred for 60 min. (precipitation of sodium salt). 67.9 mg (0.537 mmol) chloromethyldimethylphosphanoxide were added, the mixture stirred overnight to give a clear solution and evaporated. The remaining mixture was given to water and extracted with ethyl acetate. The organic phase was dried (Na₂SO₄) and evaporated. Separation of the residue by HPLC/MS on a OJ-chiral column gave 80 mg of the title compound and 25 mg of 1-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-1H-[1,2,3]triazole (example 1.17).

MS: 575.4(ESI+)

¹H-NMR(500 MHz, CDCl₃): δ=1.53(d, 6H, CH₃), 1.65(m, 4H, CH₂—CH₂—Ar, CH₂—CH₂-triazole), 2.59(t, 2H, CH₂—Ar), 2.69(t, 2H, CH₂-triazole), 4.86(d, 2H, NCH₂), 5.02(s, 2H, OCH₂), 6.92(d, 3H, 3′-/5′-H, vinyl-H), 7.06(d, 2H, 2′-/6′-H), 7.23(d, 2H, ArOCF₃), 7.39(triazole), 7.55(d, J=16.7 Hz, 1H, vinyl-H), 7.56(d, 2H, ArOCF₃), 7.68(s, 1H, oxazole).

### Example 1.19

### 4-(2-{4-[4-(4-{2-[(E)-2-(4-Trifluoromethoxy-phenyl)-vinyl]-oxazol-4-ylmethoxy}-phenyl)-butyl]-[1,2,3]triazol-2-yl}-ethyl)-morpholine

22.4 mg (0.89 mmol) 95% sodium hydride were added to a solution of 200 mg (0.413 mmol) 4-[4-(4-{2-[(E)-2-(4-Trifluoromethoxy-phenyl)-vinyl]-oxazol-4-ylmethoxy}-phenyl)-butyl]-1H-[1,2,3]triazole in 6.0 ml DMF and stirred for 3 days. 81 mg (0.43 mmol) N-(2-chloroethyl)-morpholine hydrochloride were added, the mixture stirred overnight to give a clear solution and evaporated. Separation of the residue by HPLC/MS gave 109 mg of the title compound and 68 mg that were further purified on a OJ-chiral column (methanol/water 99:1) to yield 27 mg 4-(2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine (example 1.20) and 17 mg 4-(2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine (example 1.21).

MS: 598.2(ESI+)

¹H-NMR(500 MHz, D₆DMSO): δ=1.59(m, 4H, CH₂—CH₂—Ar, CH₂—CH₂-triazole), 2.37(m, 4H, NCH₂-morpholine), 2.54(t, 2H, NCH₂-morpholine), 2.63(t, 2H, CH₂—Ar), 2.79(t, 2H, CH₂-triazole), 3.50(t, 4H, CH₂O), 4.44(t, 2H, CH₂N), 4.98(s, 2H, OCH₂), 6.94(d, 2H, 2′-/6′-H), 7.11(d, 2H, 3′-/5′-H), 7.20(d, J=16.7 Hz, 1H, vinyl-H), 7.40(d, 2H, ArOCF₃), 7.51(s, 1H, triazole), 7.57(d, J=16.7 Hz, 1H, vinyl-H), 7.87(d, 2H, ArOCF₃), 8.19(s, 1H, oxazole).

### Example 1.20

### 4-(2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine

¹H-NMR(500 MHz, CDCl₃): δ=1.5-1.7(m, 4H, CH₂—CH₂—Ar, CH₂—CH₂-triazole), 2.48(m, 4H, NCH₂-morpholine), 2.60(t, 2H, CH₂—Ar), 2.74(t, 2H, NCH₂-morpholine), 2.80(t, 2H, CH₂-triazole), 3.68(t, 4H, CH₂O), 4.42(t, 2H, CH₂N), 5.01(s, 2H, OCH₂), 6.91(m, 3H, 2′-/6′-H, vinyl-H), 7.09(d, 2H, 3′-/5′-H), 7.23(d, 2H, ArOCF₃), 7.35(s, 1H, triazole), 7.51(d, J=16.4 Hz, 1H, vinyl-H), 7.54(d, 2H, ArOCF₃), 7.66(s, 1H, oxazole).

### Example 1.21

### 4-(2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine

¹H-NMR(500 MHz, CDCl₃). δ=1.75(m, 4H, CH₂—CH₂—Ar, CH₂—CH₂-triazole), 2.49(m, 4H, NCH₂-morpholine), 2.64(t, 2H, CH₂—Ar), 2.68(t, 2H, NCH₂-morpholine), 2.84(t, 2H, CH₂-triazole), 3.68(t, 4H, CH₂O), 4.34(t, 2H, CH₂N), 5.04(s, 2H, OCH₂), 6.93(d, J=16.4, 1H, vinyl-H), 6.95(d, 2H, 2′-/6′-H), 7.11(d, 2H, 3′-/5′-H), 7.26(d, 2H, ArOCF₃), 7.43(s, 1H, triazole), 7.53(d, J=16.4 Hz, 1H, vinyl-H), 7.57(d, 2H, ArOCF₃), 7.69(s, 1H, oxazole).

### Example 1.22

### 3-{4-[4-(4-{2-[(E)-2-(4-Trifluoromethoxy-phenyl)-vinyl]-oxazol-4-ylmethoxy}-phenyl)-butyl]-[1,2,3]triazol-2-yl}-propan-1-ol

13.6 mg (0.54 mmol) 95% sodium hydride were added to a solution of 200 mg (0.413 mmol) 4-[4-(4-{2-[(E)-2-(4-Trifluoromethoxy-phenyl)-vinyl]-oxazol-4-ylmethoxy}-phenyl)-butyl]-1H-[1,2,3]triazole in 6.0 ml DMF and stirred for 60 min. (precipitation of sodium salt). 75 mg (0.54 mmol) 3-bromo-1-propanol were added, the mixture stirred overnight to give a clear solution and evaporated. The remaining mixture was given to water and extracted with ethyl acetate. The organic phase was dried (Na₂SO₄) and evaporated. Separation of the residue by HPLC/MS gave 42 mg of the title compound and 37 mg that were further purified on a OJ-chiral column (methanol/water 85:15) to yield 8 mg 3-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-propan-1-ol (example 1.23) and 7 mg 3-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-propan-1-ol (example 1.24).

MS: 543.29(ESI+)

¹H-NMR(500 MHz, CDCl₃): δ=1.73(m, 4H, N—CH₂—CH₂—CH₂—O, CH₂—CH₂—Ar), 2.12(quintet, 2H, CH₂—CH₂-triazole), 2.59(t, 2H, CH₂—Ar), 2.68(t, 2H, CH₂-triazole), 3.62(t, 2H, CH₂OH), 4.51(t, 2H, CH₂N), 5.01(s, 2H, OCH₂), 6.90(m, 3H, 2′-/6′-H, vinyl-H), 7.09(d, 2H, 340 -/5′-H), 7.22(d, 2H, ArOCF₃), 7.32(s, 1H, triazole), 7.50(d, J=16.7 Hz, 1H, vinyl-H), 7.54(d, 2H, ArOCF₃), 7.65(s, 1H, oxazole).

### Example 1.23

### 3-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-propan-1-ol

MS: 556.26(M+Na, ESI+)

¹H-NMR(500 MHz, CDCl₃): δ=1.72(m, 4H, N—CH₂—CH₂—CH₂—O, CH₂—CH₂—Ar), 2.12(quintet, 2H, CH₂—CH₂-triazole), 2.62(t, 2H, CH₂—Ar), 2.74(t, 2H, CH₂-triazole), 3.66(t, 2H, CH₂OH), 4.49(t, 2H, CH₂N), 5.03(s, 2H, OCH₂), 6.93(m, 3H, 2′-/6′-H, vinyl-H), 7.12(d, 2H, 3′-/5′-H), 7.26(d, 2H, ArOCF₃), 7.28(s, 1H, triazole), 7.53(d, J=16.4 Hz, 1H, vinyl-H), 7.57(d, 2H, ArOCF₃), 7.68(s, 1H, oxazole).

### Example 1.24

### 3-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-propan-1-ol

MS: 556.2(M+Na, ESI+)

¹H-NMR(500 MHz, CDCl₃): δ=1.71(m, 4H, N—CH₂—CH₂—CH₂—O, CH₂—CH₂—Ar), 2.09(quintet, 2H, CH₂—CH₂-triazole), 2.6(m, 4H, CH₂—Ar, CH₂-triazole), 3.64(t, 2H, CH₂OH), 4.37(t, 2H, CH₂N), 5.04(s, 2H, OCH₂), 6.94(d, J=16.4 Hz, 1H, 3H, vinyl-H), 6.95(d, 2H, 2′-/6′-H), 7.11(d, 2H, 3′-/5′-H), 7.26(d, 2H, ArOCF₃), 7.46(s, 1H, triazole), 7.55(d, J=16.4 Hz, 1H, vinyl-H), 7.57(d, 2H, ArOCF₃), 7.69(s, 1H, oxazole).

### Example 2.1

### 5-[4-(4-{2-[(E)-2-(4-Trifluoromethoxy-phenyl)-vinyl]-oxazol-4-ylmethoxy}-phenyl)-butyl]-2H-tetrazole

### 4-(4-2H-tetrazol-5-yl-butyl)-phenol

450 mg (1.94 mmol) 5-(4-(4-methoxy-phenyl)-butyl)-2H-tetrazole and 1.5 ml 48% aqueous hydrobromic acid were stirred at 80° C. for 17 h. The reaction mixture was adjusted to pH=4 by addition of conc. NaOH and the aqueous phase discarded. Purification of the undissolved residue by HPLC-MS (methanol/water 7:3, pH=2.3) gave 220 mg (52%) of the title compound.

MS: M=219.3(APCI+), 217.3(APCI−).

¹H-NMR(400 MHz, D₆-DMSO): δ=1.53(quintet, 2H, CH₂—CH₂—Ar), 1.68(quintet, 2H, CH₂—CH₂-tetrazole), 2.48(t, 2H, CH₂—Ar), 2.89(t, 2H, CH₂-tetrazole), 6.65(d, 2H, 2′-6′-H), 6.69(d, 2H, 3′-/5′-H), 9.1(br, 1H, OH), 16(br, 1H, NH).

### 5-[4-(4-{2-[(E)-2-(4-trifluoromethoxy-phenyl)-vinyl]-oxazol-4-ylmethoxy}-phenyl)-butyl]-2H-tetrazole

15.7 mg (0.62 mmol) 95% sodium hydride were given at 0° C. to a solution of 66 mg (0.30 mmol) 4-(4-tetrazol-5-yl-butyl)-phenol in 4.0 ml N,N-dimethylformamide and stirred for 15 min. 92 mg (0.30 mmol) 4-chloromethyl-2-[2-(4-trifluoromethoxy-phenyl)-vinyl]-oxazole were added and stirring continued at 25° C. for 2 h. The reaction mixture was neutralized with HCl, poured into water and the resulting precipitate was purified by HPLC-MS (methanol/water 8:2).

Yield: 50 mg (34%).

MS: M=485.2(EI), 486.2(ESI+), 484.2(ESI−).

¹H-NMR(400 MHz, D₆-DMSO): δ=1.58(quintet, 2H, CH₂—CH₂—Ar), 1.69(quintet, 2H, CH₂—CH₂-tetrazole), 2.56(t, 2H, CH₂—Ar), 2.91(t, 2H, CH₂-tetrazole), 4.98(s, 2H, OCH₂), 6.96(d, 2H, 2′-/6′-H), 7.11(d, 2H, 3′-/5′-H), 7.21(d, J=16.4 Hz, 1H, vinyl-H), 7.41(d, 2H, ArOCF₃), 7.57(d, J=16.4 Hz, 1H, vinyl-H), 7.86(d, 2H, ArOCF₃), 8.20(s, 1H, oxazole).

### Example 2.2

### Methyl-5-[4-(4-{2-[(E)-2-(4-trifluoromethoxy-phenyl)-vinyl]-oxazol-4-ylmethoxy}-phenyl)-butyl]-1H-tetrazole

5.3 mg (0.13 mmol) 60% sodium hydride were added to a solution of 61 mg (0.13 mmol) 5-[4-(4-[2-[(E)-2-(4-trifluoromethoxy-phenyl)-vinyl]-oxazol-4-ylmethoxy}-phenyl]-2H-tetrazole in 1.0 ml DMF and stirred for 10 min. 19 mg (0.13 mmol) iodomethane were added, the mixture stirred overnight and evaporated. Separation by HPLC-MS gave 10 mg of the title compound and 10 mg of 2-Methyl-5-[4-(4-{2-[(E)-2-(4-trifluoromethoxy-phenyl)-vinyl]-oxazol-4-ylmethoxy}-phenyl)-butyl]-2H-tetrazole (example 2.2).

MS: M=499.2(EI), 500.1(ESI+)

¹H-NMR(500 MHz, D₄-CH₃OH): δ=1.71(quintet, 2H, CH₂—CH₂—Ar), 1.81(quintet, 2H, CH₂—CH₂-tetrazole), 2.63(t, 2H, CH₂—Ar), 2.90(t, 2H, CH₂-tetrazole), 4.01(s, 3H, NCH₃), 5.02(s, 2H, OCH₂), 6.95(d, 2H, 2′-/6′-H), 7.08(d, J=16.4 Hz, 1H, vinyl-H), 7.15(d, 2H, 3′-/5′-H), 7.34(d, 2H, ArOCF₃), 7.60(d, J=16.4 Hz, 1H, vinyl-H), 7.76(d, 2H, ArOCF₃), 7.97(s, 1H, oxazole).

### Example 2.3

### Methyl-5-[4-(4-{2-[(E)-2-(4-trifluoromethoxy-phenyl)-vinyl]-oxazol-4-ylmethoxy}-phenyl)-butyl]-2H-tetrazole

5.3 mg (0.13 mmol) 60% sodium hydride were added to a solution of 61 mg (0.13 mmol) 5-[4-(4-{2-[(E)-2-(4-trifluoromethoxy-phenyl)-vinyl]-oxazol-4-ylmethoxy}-phenyl)-butyl]-2H-tetrazole in 1.0 ml DMF and stirred for 10 min. 19 mg (0.13 mmol) iodomethane were added, the mixture stirred overnight and evaporated. Separation by HPLC-MS gave 10 mg of the title compound and 10 mg of 1-Methyl-5-[4-(4-{2-[(E)-2-(4-trifluoromethoxy-phenyl)-vinyl]-oxazol-4-ylmethoxy}-phenyl)-butyl]-1H-tetrazole (example 2.3).

MS: M=499.2(EI), 500.1(ESI+)

¹H-NMR(500 MHz, D₄-CH₃OH): δ=1.67(quintet, 2H, CH₂—CH₂—Ar), 1.78(quintet, 2H, CH₂—CH₂-tetrazole), 2.65(t, 2H, CH₂—Ar), 2.92(t, 2H, CH₂-tetrazole), 4.35(s, 3H, NCH₃), 5.02(s, 2H, OCH₂), 6.94(d, 2H, 2′-/6′-H), 7.08(d, J=16.4 Hz, 1H, vinyl-H), 7.13(d, 2H, 3′-/5′-H), 7.34(d, 2H, ArOCF₃), 7.62(d, J=16.4 Hz, 1H, vinyl-H), 7.77(d, 2H, ArOCF₃), 7.97(s, 1H, oxazole).

### Example 2.4

### 2-{5-[4-(4-{2-[2-(4-Trifluoromethoxy-phenyl)-vinyl]-oxazol-4-ylmethoxy}-phenyl)-butyl]-tetrazol-1-yl}-ethanol

12.5 mg (0.519 mmol) 95% sodium hydride were added to a solution of 240 mg (0.494 mmol) 5-[4-(4-{2-[(E)-2-(4-trifluoromethoxy-phenyl)-vinyl]-oxazol-4-ylmethoxy}-phenyl)-butyl]-2H-tetrazole in 4.0 ml DMF and stirred for 15 min. 68 mg (0.544 mmol) 2-bromoethanol were added, the mixture stirred overnight and evaporated. Separation by LC-MS (methanol/water 8:2, pH=2.3) on a RP18 column gave 40 mg of the title compound and 110 mg of 2-{5-[4-(4-{2-[2-(4-trifluoromethoxy-phenyl)-vinyl]-oxazol-4-ylmethoxy}-phenyl-butyl]-tetrazol-2-yl}-ethanol (example 2.5).

MS: M=530.1(EI)

¹H-NMR(500 MHz, D₆-DMSO): δ=1.64(quintet, 2H, CH₂—CH₂—Ar), 1.74(quintet, 2H, CH₂—CH₂-tetrazole), 2.57(t, 2H, CH₂—Ar), 2.90(t, 2H, CH₂-tetrazole), 3.76(q, 2H, CH₂OH), 4.36(t, 2H, CH₂N), 4.98(s, 2H, OCH₂), 5.01(t, 1H, OH), 6.94(d, 2H, 2′-/6′-H), 7.14(d, 2H, 3′-/5′-H), 7.20(d, J=16.4 Hz, 1H, vinyl-H), 7.39(d, 2H, ArOCF₃), 7.56(d, J=16.4 Hz, 1H, vinyl-H), 7.88(d, 2H, ArOCF₃), 8.19(s, 1H, oxazole).

### Example 2.5

### 2-{5-[4-(4-{2-[2-(4-Trifluoromethoxy-phenyl)-vinyl]-oxazol-4-ylmethoxy}-phenyl)-butyl]-tetrazol-2-yl}-ethanol

12.5 mg (0.519 mmol) 95% sodium hydride were added to a solution of 240 mg (0.494 mmol) 5-[4-(4-{2-[(E)-2-(4-trifluoromethoxy-phenyl)-vinyl]-oxazol-4-ylmethoxy}-phenyl)-butyl]-2H-tetrazole in 4.0 ml DMF and stirred for 15 min. 68 mg (0.544 mmol) 2-bromoethanol were added, the mixture stirred overnight and evaporated. Separation by LC-MS (methanol/water 8:2, pH=2.3) on a RP18 column gave 110 mg of the title compound and 40 mg of 2-{5-[4-(4-{2-[2-(4-trifluoromethoxy-phenyl)-vinyl]-oxazol-4-ylmethoxy}-phenyl)-butyl]-tetrazol-1-yl}-ethanol (example 2.4).

MS: M=530.2(EI)

¹H-NMR(500 MHz, D₄-CH₃OH): δ=1.71(quintet, 2H, CH₂—CH₂—Ar), 1.80(quintet, 2H, CH₂—CH₂-tetrazole), 2.62(t, 2H, CH₂—Ar), 2.90(t, 2H, CH₂-tetrazole), 4.06(t, 2H, CH₂OH), 4.70(t, 2H, CH₂N), 5.00(s, 2H, OCH₂), 6.93(d, 2H, 2′-/6′-H), 7.05(d, J=16.4 Hz, 1H, vinyl-H), 7.12(d, 2H, 3′-/5′-H), 7.32(d, 2H, ArOCF₃), 7.59(d, J=16.4 Hz, 1H, vinyl-H), 7.75(d, 2H, ArOCF₃), 7.94(s, 1H, oxazole).

### Example 3.1

### 4-[4-(2-Methyl-4-{2-[(E)-2-(4-Trifluoromethoxy-phenyl)-vinyl]-oxazol-4-ylmethoxy}-phenyl)-butyl]-1H-[1,2,3]triazole

### 1-(4-Bromo-butyl)-4-methoxy-2-methylbenzene

After starting the Grignard reaction by adding 25.0 ml 4-bromo2-methyl-anisole to a mixture of 30.2 g (1.24 mol) magnesium turnings and 650 ml THF, 150 ml 4-bromo2-methylanisole (total: 175.0 ml were added at a pace sufficient to maintain reflux temperature. The reaction mixture was heated to reflux for additional 5 h, cooled to r.t. and dropped at 0° C. within 1 h to a stirred solution prepared by mixing 904 g (500 ml, 4.19 mol) 1,4-dibromo-butane in 1000 ml THF with a freshly prepared solution of 1.28 g (60.0 mmol) LiCl and 4.00 g (29.8 mmol) Cu(II)Cl₂ in 140 ml THF. Stirring was continued for 12 h at r.t. followed by the addition of 400 ml of a 20% ammonium chloride solution and 200 ml ethyl acetate. The water phase was extracted twice with 250 ml ethyl acetate, all organic phases were combined, dried over sodium sulphate and evaporated. The resulting oil was fractionated by vacuum distillation. Yield: 226.3 g (71%), b.p. 131-134° C./0.01 mbar.

¹H-NMR(400 MHz, D₆-DMSO): δ=1.60(quintet, 2H, CH₂—CH₂-Ph), 1.82(quintet, 2H, CH₂—CH₂—Br), 2.23(s, 3H, CH₃), 2.51(t, 2H, CH₂-Ph), 3.55(t, 2H, CH₂—Br), 3.70(s, 3H, OCH₃), 6.67d, 1H, 5-H), 6.72(s, 1H, 3-H), 7.01(d, 1H, 6-H).

### 1-(4-Iodo-butyl)-4-methoxy-2-methyl-benzene

A mixture consisting of 70.0 g (272 mmol) 1-(4-bromo-butyl)-4-methoxy-2-methyl-benzene, 40.8 g (272 mmol) sodium iodide and 1100 ml acetone was heated to reflux temperature for 1 h. The resulting suspension was cooled to r.t. and the precipitated sodium bromide removed by filtration. The filtrate was stripped off the solvents by vacuum distillation and the residue distributed between water and diethyl ether. After drying of the organic phase over sodium sulphate, vacuum distillation gave 75.3 g (91%) of the title compound as slightly yellow coloured liquid.

MS: M=304.2 (ESI).

¹H-NMR(400 MHz, CDCl₃): δ=1.67(quintet, 2H, CH₂—CH₂—Ar), 1.88(quintet, 2H, CH₂—CH₂—I), 2.28(s, 3H, CH₃), 2.56(t, 2H, CH₂—Ar), 3.21(t, 2H, CH₂—I), 3.77(s, 3H, OCH₃), 6.68(d, 1H, 5-H), 6.70(s, 1H, 3-H), 7.02(d, 1H, 6-H).

### [6-(4-Methoxy-2-methyl-phenyl)-hex-1-ynyl]-trimethyl-silane

198 ml (495 mmol) of 2.5 M butyllithium in n-hexane was added dropwise at −78° C. to a solution of 48.6 g (70.0 ml, 495 mmol) trimethylsilylacetylene and 59.9 ml (495 mmol) DMPU in 700 ml THF. After stirring for 1 h at −78° C. a solution of 75.3 g (248 mmol) 1-(4-Iodo-butyl)-4-methoxy-2-methyl-benzene in 260 ml THF was added at −78° C. and stirring continued for 30 min. The reaction mixture was allowed to warm to r.t. overnight and then hydrolysed by a saturated ammonium chloride solution. The water phase was extracted with ether and the combined organic phases were dried over sodium sulphate. Removal of solvents in vacuo gave 87.1 g yellow liquid, which still contained solvent and was used without further purification.

¹H-NMR(400 MHz, CDCl₃): δ=0.15(s, 9H, Si(CH₃)₃), 1.5-1.7(m, 4H, CH₂—CH₂—C≡CH), CH₂—CH₂—Ar), 2.23(t, 2H, CH₂—C≡CH), 2.25(CH₃), 2.53(t, 2H, CH₂—Ar), 3.77(s, 3H, OCH₃), 6.65(d, 1H, 5′-H) 6.69(s, 1H, 3′-H), 7.05(d, 1H, 6′-H)

### 1-Hex-5-ynyl-4-methoxy-2-methyl-benzene

A mixture of 87.1 g (317 mmol) [6-(4-methoxy-2-methyl-phenyl)-hex-1-ynyl]-trimethyl-silane, 2300 ml methanol and 317 ml (635 mmol) 2N NaOH was stirred for 2 h at r.t. After neutralization with 317 ml 2N HCl methanol was distilled off and the aqueous phase extracted with diethyl ether. Drying (Na₂SO₄) and removal of solvents in vacuo gave 59.9 g (93%) of the title compound.

¹H-NMR(400 MHz, CDCl₃) δ=1.55(quintet, 2H, CH₂—CH₂—C≡C), 1.61(quintet, 2H, CH₂—CH₂—Ar), 1.88(s, 1H, ≡CH), 2.21(t, 2H, CH₂—C≡C), 2.28(s, 3H, CH₃), 2.55(t, 2H, CH₂—Ar), 3.78(s, 3H, OCH₃), 6.65(d, 1H, 5′-H) 6.69(s, 1H, 3′-H), 7.05(d, 1H, 6′-H)

### 1-Benzyl-4-(4-(4-Methoxy-2-methyl-phenyl)-butyl)-1H-[1,2,3]triazole

34.9 g (172 mmol) 1-hex-5-ynyl-4-methoxy-2-methyl-benzene and 23.0 g (21.6 ml, 172 mmol) benzylazide were dissolved in 813 ml t-butanol/water 1:1. A solution of 3.48 g (17.6 mmol) sodium ascorbate in 30 ml water and then 464 mg (1.9 mmol) copper(II)sulphate in 20 ml water was added. Stirring was continued for 12 h. After addition of 1000 ml water the separated oil was separated and purified by HPLC on a RP18/PRO C18 column (methanol/water 7:3, pH=2.3). Yield: 41.7 g (72%) oil that crystallized upon standing.

¹H-NMR(400 MHz, CDCl₃): δ=1.57(quintet, 2H, CH₂—CH₂—Ar), 1.71(quintet, 2H, CH₂—CH₂-triazole), 2.28(s, 3H, CH₃), 2.53(t, 2H, CH₂—Ar), 2.71(t, 2H, CH₂-triazole), 3.75(s, 3H, OCH₃), 5.46(NCH₂), 6.64(d, 1H, 5′-H) 6.68(s, 1H, 3′-H), 6.99(d, 1H, 6′-H), 7.16(s, 1H, triazole), 7.24(m, 2H, benzyl-H), 7.36(m, 3H, benzyl-H)

### 4-(4-(4-Methoxy-2-methyl-phenyl)-butyl)-1H-[1,2,3]triazole

A solution of 17.5 g (52.2 mmol) 1-Benzyl-4-(4-(4-Methoxy-2-methyl-phenyl)-butyl)-1H-[1,2,3]triazole in 100 ml methanol was hydrogenated over Pd/C for 6 h at 120° C. and 150 ba After filtration from the catalyst and removal of solvents 11.2 (88%) of the title compound was obtained as oil.

MS: 246.3(ESI+)

¹H-NMR(400 MHz, CDCl₃): δ=1.59(quintet, 2H, CH₂—CH₂—Ar), 1.75(quintet, 2H, CH₂—CH₂-triazole), 2.14(s, 3H, CH₃), 2.46(t, 2H, CH₂—Ar), 2.65(t, 2H, CH₂-triazole), 3.77(s, 3H, OCH₃), 6.48(d, 1H, 5′-H), 6.52(s, 1H, 3′-H), 6.86(d, 1H, 6′-H), 7.53(s, 1H, triazole), 8.16(br, 1H, NH).

### 3-Methyl-4-[4-(3H-[1,2,3]triazole-4-yl)butyl]-phenol

A mixture of 4-(4-(4-methoxy-2-methyl-phenyl)-butyl)-1H-[1,2,3]triazole and 45 ml 48% hydrobromic acid was stirred at 100° C. for 6 h. After adjustment to pH=6 by addition of conc. sodium hydroxide solution, the aqueous layer was discarded and the remaining oil purified by HPLC-MS (RP18, methanol/water 7:3, pH=2.3). Yield 4.33 g (36%).

MS: M=232.26(APCI+).

¹H-NMR(400 MHz, D₆-DMSO): δ=1.47(quintet, 4H, CH₂—CH₂—Ar), 1.63(quintet, 2H, CH₂—CH₂-triazole), 2.14(s, 3H, CH₃), 2.46(t, 2H, CH₂—Ar), 2.65(t, 2H, CH₂-triazole), 6.48(d, 1H, 5′-H), 6.52(s, 1H, 3′-H), 6.86(d, 1H, 6′-H), 7.53(s, 1H, triazole), 8.96(br, 1H, NH).

### 3-Methyl-4-[4-(1-trityl-1H-[1,2,3]triazole-4-yl)-butyl]-phenol

A solution of 6.26 g (22.5 mmol) triphenylchloromethane in 100 ml DMF was added at 0° C. to a solution of 4.33 g (18.7 mmol) 2-Methyl-4-(4-1H-[1,2,3]triazol-4-yl-butyl)-phenyl and 2.27 g (22.5 mmol) triethylamine in 20 ml DMF. The mixture was allowed to reach r.t. overnight and solvents were removed in vacuo. After distribution of the residue between water and ethyl acetate, the organic phase was dried (sodium sulphate), solvents distilled off and the residue purified by column chromatography on silica gel (heptane/ethyl acetate 2:1). Yield 3.50 g (40%).

¹H-NMR(400 MHz, D₆-DMSO): δ=1.43(quintet, 2H, CH₂—CH₂—Ar), 1.62(quintet, 2H, CH₂—CH₂-triazole), 2.11(s, 3H, CH₃), 2.43(t, 2H, CH₂—Ar), 2.65(t, 2H, CH₂-triazole), 6.47(d, 1H, 5′-H), 6.51(s, 1H, 3′-H), 6.83(d, 1H, 6′-H), 7.01(m, 6H, trityl), 7.44(s, 1H, triazole), 7.38(m, 6H, trityl), 8.96(s, 1H, OH).

### 4-[4-(2-Methyl-4-{2-[(E)-2-(4-trifluoromethoxy-phenyl)-vinyl]-oxazol-4-ylmethoxy}-phenyl)-butyl]-1-trityl-1H-[1,2,3]triazole

200 mg (7.92 mmol) 95% sodium hydride were given at 0° C. to a solution of 3.54 g (7.47 mmol) 3-Methyl-4-[4-(1-trityl-1H-[1,2,3]triazol-4-yl)-butyl]-phenol in 30 ml N,N-dimethylformamide and stirred for 30 min. 2.27 g (7.47 mmol) 4-chloromethyl-2-[2-(4-trifluoromethoxy-phenyl)-vinyl]-oxazole in 10 ml DMF were added and stirring continued overnight at 25° C. The reaction mixture was poured into water, after standing for 2 h the precipitate was isolated by filtration, washed with water and dried to yield 4.50 g (81%) of the title compound, m.p. 135-136° C.

¹H-NMR(400 MHz, D₆-DMSO): δ=1.46(quintet, 2H, CH₂—CH₂—Ar), 1.62(quintet, 2H, CH₂—CH₂-triazole), 2.18(s, 3H, CH₃), 2.49(t, 2H, CH₂—Ar), 2.67(t, 2H, CH₂-triazole), 4.96(s, 2H, OCH₂), 6.77(d, 1H, 5′-H), 6.80(s, 1H, 3′-H), 6.83(d, 1H, 6′-H), 7.03(m, 7.03(m, 7H, trityl-H, 6′-H), 7.21(d, 1H, vinyl-H), 7.4(m, 11H, trityl-H, ArOCF₃), 7.46(s, 1H, triazole), 7.56(d, 1H, vinyl-H), 7.86(d, 2H, ArOCF₃), 8.20(s, 1H, oxazole).

### 4-[4-(2-Methyl-4-{2-[(E)-2-(4-trifluoromethoxy-phenyl)-vinyl]-oxazol-4-ylmethoxy}-phenyl)-butyl]-1H-[1,2,3]triazole

A mixture of 3.91 g (5.28 mmol) 4-[4-(2-methyl-4-{2-[(E)-2-(4-Trifluoromethoxy-phenyl)-vinyl]-oxazol-4-ylmethoxy}-phenyl)-butyl]-1-trityl-1H-[1,2,3]triazole, 8 ml formic acid and 40 ml THF was stirred at 80° C. for 36 h. The reaction mixture was added to water, neutralized with 2 N sodium hydroxide to pH=6 and extracted with ethyl acetate. The organic phase was dried (Na₂SO₄), solvents distilled off in vacuo and the residue purified by chromatography on silica (heptane/ethyl acetate 2:1) to give 0.89 g (34%) of the title compound.

MS: M=499.1(APCI+), 497.1(APCR−).

¹H-NMR(400 MHz, D₆-DMSO): δ=1.51(quintet, 2H, CH₂—CH₂—Ar), 1.65(quintet, 2H, CH₂—CH₂-triazole), 2.22(s, 3H, CH₃), 2.51(t, 2H, CH₂—Ar), 2.67(t, 2H, CH₂-triazole), 4.96(s, 2H, —OCH₂—), 6.77(d, 1H, 5′-H), 6.81(s, 1H, 3′-H), 7.02(d, 1H, 6′-H), 7.21(d, 1H, vinyl-H), 7.40(d, 2H, 3″-/5″-H, ArOCF₃), 7.56(d, 1H, vinyl-H), 7.58(s, 1H, triazole), 7.86(d, 2H, 2″-/6″-H, ArOCF₃), 8.19(s, 1H, oxazole), 14.6(br, 1H, NH).

### Example 3.2

### Methyl-4-[4-(2-methyl-4-{2-[(E)-2-(4-trifluoromethoxy-phenyl)-vinyl]-oxazol-4-ylmethoxy}-phenyl)-butyl]-2H-[1,2,3]triazole

16.5 mg (0.65 mmol) 95% sodium hydride were added to a solution of 250 mg (0.502 mmol) 4-[4-(2-methyl-4-{2-[(E)-2-(4-Trifluoromethoxy-phenyl)-vinyl]-oxazol-4-ylmethoxy}-phenyl)-butyl]-1H-[1,2,3]triazole in 6.0 ml DMF and stirred for 60 min. (precipitation of sodium salt). 95.5 mg (41 μl, 0.65 mmol) iodomethane were added, the mixture stirred overnight to give a clear solution and evaporated. The remaining mixture was given to water and extracted with ethyl acetate. The organic phase was dried (Na₂SO₄) and evaporated. Separation of the residue by HPLC/MS gave 100 mg of 2-Methyl-4-[4-(2-methyl-4-{2-[(E)-2-(4-trifluoromethoxy-phenyl)-vinyl]-oxazol-4-ylmethoxy}-phenyl)-butyl]-2H-[1,2,3]triazole (example 3.2) and 95 mg that were further separated on a OJ-chiral column (methanol) to yield 31 mg of 1-Methyl-4-[4-(2-methyl-4-{2-[(E)-2-(4-trifluoromethoxy-phenyl)-vinyl]-oxazol-4-ylmethoxy}-phenyl)-butyl]-1H-[1,2,3]triazole (example 3.3) and 32 mg of 1-Methyl-5-[4-(2-methyl-4-{2-[(E)-2-(4-trifluoromethoxy-phenyl)-vinyl]-oxazol-4-ylmethoxy}-phenyl)-butyl]-1H-[1,2,3]triazole (example 3.4).

MS: 513.3(APCR+)

¹H-NMR(500 MHz, CDCl₃): δ=1.64(quintet, 2H, CH₂—CH₂—Ar), 1.75(quintet, 2H, CH₂—CH₂-triazole), 2.29(s, 3H, CH₃), 2.60(t, 2H, CH₂—Ar), 2.72(t, 2H, CH₂-triazole), 4.15(s, 3H, NCH₃), 5.03(s, 2H, OCH₂), 6.78(d, 1H, 5′-H), 6.82(s, 1H, 3′-H), 6.93(d, 1H, vinyl-H), 7.05(d, 1H, 6′-H), 7.26(d, 2H, 3″-/5″-H, ArOCF₃), 7.34(s, 1H, triazole), 7.53(d, 1H, vinyl-H), 7.57(d, 2H, 2″-/6″-H, ArOCF₃), 7.67(s, 1H, oxazole).

### Example 3.3

### Methyl-4-[4-(2-methyl-4-{2-[(E)-2-(4-trifluoromethoxy-phenyl)-vinyl]-oxazol-4-ylmethoxy}-phenyl)-butyl]-1H-[1,2,3]triazole

MS: 513.3(APCR+)

¹H-NMR(500 MHz, CDCl₃): δ=1.64(quintet, 2H, CH₂—CH₂—Ar), 1.76(quintet, 2H, CH₂—CH₂-triazole), 2.29(s, 3H, CH₃), 2.60(t, 2H, CH₂—Ar), 2.77(t, 2H, CH₂-triazole), 4.07(s, 3H, NCH₃), 5.03(s, 2H, OCH₂), 6.78(d, 1H, 5′-H), 6.82(s, 1H, 3′-H), 6.93(d, 1H, vinyl-H), 7.06(d, 1H, 6′-H), 7.26(d, 2H, 3″-/5″-H, ArOCF₃), 7.28(s, 1H, triazole), 7.53(d, 1H, vinyl-H), 7.57(d, 2H, 2″-/6″-H, ArOCF₃), 7.67(s, 1H, oxazole).

### Example 3.4

### 1-Methyl-5-[4-(2-methyl-4-{2-[(E)-2-(4-trifluoromethoxy-phenyl)-vinyl]-oxazol-4-ylmethoxy}-phenyl)-butyl]-1H-[1,2,3]triazole

MS: 513.3 (APCR+)

¹H-NMR(500 MHz, CDCl₃): δ=1.67(quintet, 2H, CH₂—CH₂—Ar), 1.75(quintet, 2H, CH₂—CH₂-triazole), 2.30(s, 3H, CH₃), 2.62(t, 2H, CH₂—Ar), 2.67(t, 2H, CH₂-triazole), 3.96(s, 3H, NCH₃), 5.03(s, 2H, OCH₂), 6.79(d, 1H, 5′-H), 6.83(s, 1H, 3′-H), 6.93(d, 1H, vinyl-H), 7.04(d, 1H, 6′-H), 7.27(d, 2H, 3″-/5″-H, ArOCF₃), 7.46(s, 1H, triazole), 7.53(d, 1H, vinyl-H), 7.57(d, 2H, 2″-/6″-H, ArOCF₃), 7.68(s, 1H, oxazole).

1. A compound of formula I 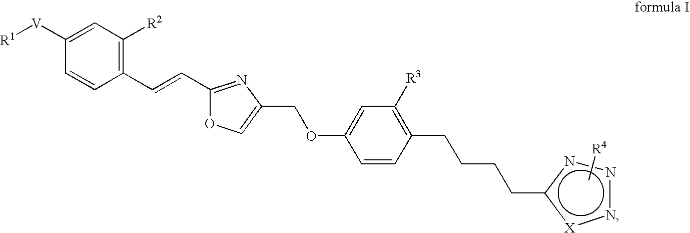
wherein
R¹ is halogenated alkyl;
R² is hydrogen or halogen;
R³ is hydrogen, alkyl or halogen;
R⁴ is hydrogen or alkyl; said alkyl being optionally substituted one or two times by —OH, —P(O)(alkyl)₂, —C(O)OH, —C(O)O-alkyl, —CN, morpholino, —S(O)-alkyl or —S(O)₂-alkyl;
V is —O— or —S—;
X is carbon or nitrogen; and a
pharmaceutically acceptable salt thereof:

2. The compound according to claim 1, wherein
R⁴ is hydrogen or alkyl; said alkyl being optionally substituted one or two times by —OH, —P(O) (alkyl)₂, —C(O)OH, —C(O)O-alkyl, —CN or morpholino.

3. The compound according to claim 1, wherein
R² is hydrogen; and
R³ is hydrogen or alkyl.

4. The compound according to claim 1, wherein
R² is hydrogen;
R³ is hydrogen; and
V is —O—.

5. The compound according to claim 1, wherein
x is carbon.

6. A compound according to claim 5 which is selected from:
4-[4-(4-{2-[(E)-2-(4-Trifluoromethoxy-phenyl)-vinyl]-oxazol-4-ylmethoxy}-phenyl)-butyl]-1H-[1,2,3]triazole;
1-Methyl-5-[4-(4-{2-[(E)-2-(4-trifluoromethoxy-phenyl)-vinyl]-oxazol-4-ylmethoxy}-phenyl)-butyl]-1H-[1,2,3]triazole;
2-Methyl-4-[4-(4-{2-[(E)-2-(4-trifluoromethoxy-phenyl)-vinyl]-oxazol-4-ylmethoxy}-phenyl)-butyl]-2H-[1,2,3]triazole;
1-Methyl-4-[4-(4-{2-[(E)-2-(4-trifluoromethoxy-phenyl)-vinyl]-oxazol-4-ylmethoxy}-phenyl)-butyl]-1H-[1,2,3]triazole;
2-{4-[4-(4-{2-[(E)-2-(4-Trifluoromethoxy-phenyl)-vinyl]-oxazol-4-ylmethoxy}-[1,2,3]triazol-2-yl}-ethanol;
2-{4-[4-(4-{2-[(E)-2-(4-Trifluoromethoxy-phenyl)-vinyl]-oxazol-4-ylmethoxy}-[1,2,3]triazol-1-yl}-ethanol;
2-{5-[4-(4-{2-[(E)-2-(4-Trifluoromethoxy-phenyl)-vinyl]-oxazol-4-ylmethoxy}-[1,2,3]triazol-1-yl}-ethanol;
3-{4-[4-(4-{2-[(E)-2-(4-Trifluoromethoxy-phenyl)-vinyl]-oxazol-4-ylmethoxy}-[1,2,3]triazol-2-yl}-propan-1-ol;
3-{4-[4-(4-{2-[(E)-2-(4-Trifluoromethoxy-phenyl)-vinyl]-oxazol-4-ylmethoxy}-[1,2,3]triazol-1-yl}-propan-1-ol; and
3-{5-[4-(4-{2-[(E)-2-(4-Trifluoromethoxy-phenyl)-vinyl]-oxazol-4-ylmethoxy}-[1,2,3]triazol-1-yl}-propan-1-ol.

7. A compound according to claim 5, which is selected from:
1-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-1H-[1,2,3]triazole;
2-(Dimethyl-phosphinoylmethyl)-4-[4-(4-{2-[(E)-2-(4-trifluoromethoxy-phenyl)-vinyl]-oxazol-4-ylmethoxy}-phenyl)-butyl]-2H-[1,2,3]triazole;
4-[4-(2-Methyl-4-{2-[(E)-2-(4-Trifluoromethoxy-phenyl)-vinyl]-oxazol-4-ylmethoxy}-phenyl)-butyl]-1H-[1,2,3]triazole;
2-Methyl-4-[4-(2-methyl-4-{2-[(E)-2-(4-trifluoromethoxy-phenyl)-vinyl]-oxazol-4-ylmethoxy}-phenyl)-butyl]-2H-[1,2,3]triazole;
1-Methyl-4-[4-(2-methyl-4-{2-[(E)-2-(4-trifluoromethoxy-phenyl)-vinyl]-oxazol-4-ylmethoxy}-phenyl)-butyl]-1H-[1,2,3 ]triazole;
1-Methyl-5-[4-(2-methyl-4-{2-[(E)-2-(4-trifluoromethoxy-phenyl)-vinyl]-oxazol-4-ylmethoxy}-phenyl)-butyl]-1H-[1,2,3]triazole;.
4-(2-{4-[4-(4-12-[(E)-2-(4-Trifluoromethoxy-phenyl)-vinyl]-oxazol-4-ylmethoxy}-phenyl)-butyl]-[1,2,3]triazol-2-yl}-ethyl)-morpholine;
4-(2-{4-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine;
4-(2-{5-[4-(4-{2-[(E)-2-(4-Trifluoromethoxy-phenyl)-vinyl]-oxazol-4-ylmethoxy}-phenyl)-butyl]-[1,2,3]triazol-1-yl}-ethyl)-morpholine; and
{4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetic acid methyl ester.

8. A compound according to claim 5 which is selected from:
{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester;
{5-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid methyl ester;
{4-[4-(4-{2-[(E)-2-(4-Trifluoromethoxy-phenyl)-vinyl]-oxazol-4-ylmethoxy}-phenyl)-butyl]-[1,2,3]triazol-2-yl}-acetonitrile;
{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetonitrile;
{5-[4-(4-{2-[(E)-2-(4-Trifluoromethoxy-phenyl)-vinyl]-oxazol-4-ylmethoxy}-phenyl)-butyl]-[1,2,3]triazol-1-yl}-acetonitrile;
{4-[4-(4-{2-[2-(4-trifluoromethoxy-phenyl)-vinyl]-oxazol-4-ylmethoxy}-phenyl)-butyl]-[1,2,3]triazol-2-yl}-acetic acid sodium salt;
{4-[4-(4-{2-[(E)-2-(4-Trifluoromethoxy-phenyl)-vinyl]-oxazol-4-ylmethoxy}-phenyl)-butyl]-[1,2,3]triazol-1-yl}-acetic acid sodium salt; and
{5-[4-(4-{2-[2-(4-Trifluoromethoxy-phenyl)-vinyl]-oxazol-4-ylmethoxy}-phenyl)-butyl]-[1,2,3]triazol-1-yl}-acetic acid sodium salt.

9. The compound according to claim 1, wherein X is nitrogen.

10. A compound according to claim 9 which is selected from:
5-[4-(4-{2-[(E)-2-(4-Trifluoromethoxy-phenyl)-vinyl]-oxazol-4-ylmethoxy}-phenyl)-butyl]-2H-tetrazole;
1-Methyl-5-[4-(4-{2-[(E)-2-(4-trifluoromethoxy-phenyl)-vinyl]-oxazol-4-ylmethoxy}-phenyl)-butyl]-1H-tetrazole;
2-Methyl-5-[4-(4-{2-[(E)-2-(4-trifluoromethoxy-phenyl)-vinyl]-oxazol-4-ylmethoxy}-phenyl)-butyl]-2H-tetrazole;
2-{5-[4-(4-{2-[(E)-2-(4-Trifluoromethoxy-phenyl)-vinyl]-oxazol-4-ylmethoxy}-phenyl)-butyl]-tetrazol-1-yl}-ethanol; and
2-{5-[4-(4-{2-[(E)-2-(4-Trifluoromethoxy-phenyl)-vinyl]-oxazol-4-ylmethoxy}-phenyl)-butyl]-tetrazol-2-yl}-ethanol.

11. A process for the manufacture of the compounds of formula I in claim 1, wherein a compound of formula V 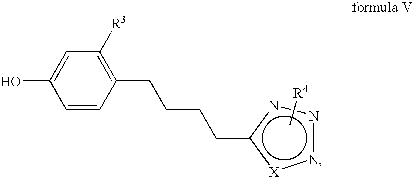
wherein R³ and R⁴ have the significance as given in formula I above in claim 1 or R⁴ is trityl,
is reacted with a compound of formula VI 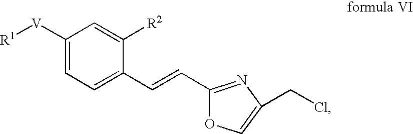
wherein R¹, R²and V have the significance given in formula I above in claim 1.

12. The process of claim 11 further comprising isolating said compound from the reaction mixture.

13. The process of claim 11 further comprising converting the compound into a pharmaceutically acceptable salt.

14. The process of claim 11 further comprising convenrting the compound into a pharmaceutically acceptable ester.

15. A pharmaceutical composition comprising one or more compounds of formula I together with a pharmaceutically acceptable excipient.

16. A method of inhibiting tumor growth comprising administering to a patient a therapeutically effective amount of a compound of claim 1.

17. A method of treating cancer comprising administering to a patient a therapeutically effective amount of a compound of claim 1.

18. The method of claim 17 wherein the cancer is human breast, colon, rectal, stomach, pancreatic, ovarian or bronchial cancer.

19. The method of claim 18, wherein the cancer is human colon cancer.
